(19) United States
(12) Patent Application Publication
(10) Pub. No.: US 2019/0307734 A1
(43) Pub. Date: Oct. 10, 2019

(54) FORMULATIONS OF ROLAPITANT

(71) Applicant: Zhuhai Beihai Biotech Co., Ltd., Zhuhai (CN)

(72) Inventor: Qun Sun, Princeton, NJ (US)

(21) Appl. No.: 16/461,196

(22) PCT Filed: Nov. 21, 2017
(86) PCT No.: PCT/US17/62798

§ 371 Date: May 15, 2019

Related U.S. Application Data
(60) Provisional application No. 62/424,805, filed on Nov. 21, 2016.

(51) Int. Cl. A61K 31/438; A61K 47/64; A61K 9/08; A61K 9/19

(52) U.S. Cl. CPC A61K 31/438; A61K 9/19; A61K 9/08; A61K 47/643

(57) ABSTRACT

This document relates to compositions comprising a non-covalently bound complex comprising rolapitant and human serum albumin, wherein the rolapitant and the human serum albumin in the composition have a ratio by weight from about 1:20 to about 1:2000. This document also relates to compositions comprising rolapitant and human serum albumin, wherein the rolapitant and the human serum albumin in the composition have a ratio by weight from about 1:20 to about 1:2000. This document also relates to compositions consisting essentially of rolapitant and human serum albumin, wherein the rolapitant and the human serum albumin in the composition have a ratio by weight from about 1:20 to about 1:2000.

### CLAIM OF PRIORITY

This application claims the benefit of U.S. provisional application No. 62/424,805 filed Nov. 21, 2016. The entire content of the foregoing is hereby incorporated by reference.

### TECHNICAL FIELD

This document relates to compositions and formulations for the treatment and prevention of chemotherapy-induced nausea and vomiting, and more particularly to compositions and formulations comprising rolapitant.

### BACKGROUND

Rolapitant is a potent, selective competitive NK1 receptor antagonist with several advantages when compared with other drugs in this class. It has a very long half-life of 180 hours (7 days), and is rapidly absorbed after oral dosing. Rolapitant (marketed under the name VARUBI) was recently approved by the US Food and Drug Admini stration for use in combination with other antiemetic agents in adults for the prevention of delayed nausea and vomiting associated with initial and repeat courses of emetogenic cancer chemotherapy, including, but not limited to, highly emetogenic chemotherapy.

In vitro studies showed that rolapitant did not inhibit CYP 2C9, 2C19, 2D6, and 3A4 enzymes or p-glycoprotein, meaning that there is a low risk of drug-drug interactions with rolapitant. Since rolapitant does not induce or inhibit the cytochrome P450 3A4 (CYP3A4) enzyme, dose adjustments of dexamethasone and certain other concomitantly administered medications metabolized by CYP34A are not required (Hesketh et al., Cancer 2016; 2418-25).

Rolapitant is practically insoluble in water. Solubility of rolapitant hydrochloride in aqueous solution is pH-dependent and is more soluble at lower pH. Rolapitant hydrochloride has good solubility in common pharmaceutical solvents such as ethanol, propylene glycol, and 40% hydroxypropyl beta cyclodextrin. Rolapitant has very low solubility at the physiological pH of 7.4 (<4 mcg/ml). Clinically, rolapitant is currently administered via the oral route in a tablet formulation to patients. Its very poor water solubility prevents its administration by the intravenous route (IV), which may be important in patients unable to take rolapitant orally. U.S. Pat. No. 9,101,615 has reported an oil-loaded micelle formulation for IV delivery of rolapitant, which contains solutol and both a medium chain and a long chain triglyceride. However, there is a medical need for suitable IV formulations of rolapitant which contain no surfactant, no oil, or no organic solvent in the IV formulations.

The compositions and methods described in the present application help meet this need.

### SUMMARY

Provided herein is a composition comprising a non-covalently bound complex comprising rolapitant and human serum albumin, wherein the rolapitant and the human serum albumin in the composition have a ratio by weight from about 1:20 to about 1:2000.

In some embodiments, the rolapitant and the human serum albumin in the composition have a ratio by weight from about 1:100 to about 1:1000, from about 1:130 to about 1:800, from about 1:140 to about 1:600, from about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free.

Also, provided herein is a composition comprising rolapitant and human serum albumin, wherein the rolapitant and the human serum albumin in the composition have a ratio by weight from about 1:20 to about 1:2000.

In some embodiments, the rolapitant and the human serum albumin in the composition have a ratio by weight from about 1:100 to about 1:1000, from about 1:130 to about 1:800, from about 1:140 to about 1:600, from about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:175, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition is a clear aqueous solution when the composition is dissolved in an aqueous solvent. In some embodiments, the aqueous solution is substantially free of solvent other than water. In some embodiments, the aqueous solution is free of solvent other than water.

In some embodiments, the composition is a clear aqueous solution for at least about 1 hour, 2 hours, 3 hours, 4 hours, 5 hours, 6 hours, 8 hours, or 24 hours, when the composition is dissolved in an aqueous solvent.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water. In some embodiments, the aqueous formulation is a clear aqueous solution for at least 1 hour, 2 hours, 3 hours, 4 hours, 5 hours, 6 hours, 8 hours, or 24 hours. In some embodiments, the solution remains clear for at least about 2 hours, 4 hours, 6 hours, 8 hours, 10 hours, 12 hours, 20 hours, 24 hours, 2 days, 3 days, 4 days, 5 days, 6 days or a week.

Also, provided herein is a pharmaceutical composition comprising the composition comprising the rolapitant and the human serum albumin as described herein, and a pharmaceutically acceptable carrier.

Also, provided herein is a method for the prevention of chemotherapy-induced nausea and vomiting or postoperative nausea and vomiting (PONV), the method comprising the step of administering to a subject in need thereof of a therapeutically effective amount of a pharmaceutical composition comprising the composition comprising the rolapitant and the human serum albumin as described herein, and a pharmaceutically acceptable carrier.

Also, provided herein is a composition consisting essentially of rolapitant and human serum albumin, wherein the rolapitant and the human serum albumin in the composition have a ratio by weight from about 1:20 to about 1:2000.

In some embodiments, the rolapitant and the human serum albumin in the composition have a ratio by weight from about 1:100 to about 1:1000, from about 1:130 to about 1:800, from about 1:140 to about 1:600, about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the solution remains clear for at least about 2 hours, 4 hours, 6 hours, 8 hours, 10 hours, 12 hours, 20 hours, 24 hours, 2 days, 3 days, 4 days, 5 days, 6 days or a week.

Also, provided herein is a pharmaceutical composition comprising the composition consisting essentially of the rolapitant and the human serum albumin as described herein, and a pharmaceutically acceptable carrier.

Also, provided herein is a method for the prevention of chemotherapy-induced nausea and vomiting or postoperative nausea and vomiting (PONV), the method comprising the step of administering to a subject in need thereof of a therapeutically effective amount of a pharmaceutical composition comprising the composition consisting essentially of the rolapitant and the human serum albumin as described herein, and a pharmaceutically acceptable carrier.

Also provided herein is a composition comprising rolapitant and human serum albumin, wherein the ratio by weight of rolapitant and the human serum albumin in the composition is from about 1:20 to about 1:2000, produced by a method comprising the steps of:

(i) obtaining an organic solution of rolapitant in a polar water-miscible organic solvent;

(ii) obtaining a first aqueous solution of human serum albumin; and

(iii) mixing the organic solution of rolapitant and the first aqueous solution of human serum albumin to obtain a second aqueous solution comprising the composition comprising rolapitant and human serum albumin.

### DETAILED DESCRIPTION

Provided herein is a composition comprising a non-covalently bound complex comprising rolapitant and human serum albumin, wherein the rolapitant and the human serum albumin in the composition have a ratio by weight from about 1:20 to about 1:2000.

In some embodiments, the rolapitant and the human serum albumin in the composition have a ratio by weight from about 1:100 to about 1:1000, from about 1:130 to about 1:800, from about 1:140 to about 1:600, about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

Provided herein is a composition comprising a non-covalently bound complex comprising rolapitant and human serum albumin, wherein the rolapitant and the human serum albumin in the complex have a ratio by weight from about 1:20 to about 1:2000. In some embodiments, the rolapitant and the human serum albumin in the composition have a ratio by weight from about 1:100 to about 1:1000, from about 1:130 to about 1:800, from about 1:140 to about 1:600, about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the non-covalent interaction between rolapitant and human serum albumin in the complex comprises hydrogen bonding. In some embodiments, the non-covalent interaction between rolapitant and human serum albumin in the complex comprises electrostatic interaction. In some embodiments, the non-covalent interaction between rolapitant and human serum albumin in the complex comprises hydrophobic interaction. In some embodiments, the non-covalent interaction between rolapitant and human serum albumin in the complex comprises Van der Waals forces. In some embodiments, the non-covalent interaction between rolapitant and human serum albumin in the complex comprises hydrogen bonding, electrostatic interaction, hydrophobic interactions and Van der Waals forces.

In some embodiments, the non-covalent interaction between rolapitant and human serum albumin in the composition comprises hydrogen bonding. In some embodiments, the non-covalent interaction between rolapitant and human serum albumin in the composition comprises electrostatic interaction. In some embodiments, the non-covalent interaction between rolapitant and human serum albumin in the composition comprises hydrophobic interaction. In some embodiments, the non-covalent interaction between rolapitant and human serum albumin in the composition comprises Van der Waals forces. In some embodiments, the non-covalent interaction between rolapitant and human serum albumin in the composition comprises hydrogen bonding, electrostatic interaction, hydrophobic interactions and Van der Waals forces.

As used herein, the term “human serum albumin” refers to native and recombinant human serum albumin. Native human serum albumin and other plasma proteins can be precipitated from human plasma by varying the pH and adding ethanol, in what is known as the Cohn fractionation process (Cohn E J et al., J. Am. Chem. Soc. 1946; 68:459-475). By controlling the pH and ethanol content, semi-purified fractions of plasma proteins can be produced. One of the last proteins to precipitate in the Cohn process is native human serum albumin. After precipitation, a wet paste of crude native human serum albumin is obtained. Subsequent bioprocessing steps (purification, filtration, pasteurization, etc.) can be used to produce a purified, stabilized form of native human serum albumin for commercial use (Lin J J et al., Pharmaceutical Research 2000; 17:391-6). Recombinant human serum albumin is a highly purified animal-, virus-, and prion-free product as alternative to native human serum albumin, to which it is structurally equivalent (Bosse D et al., J. Clin. Pharmacol. 2005; 45:57-67). Recombinant human serum albumin has been produced by various hosts, both prokaryotic and eukaryotic (Chen Z et al., Biochimica et Biophysica Acta 2013; 1830:5515-5525). A fatty acid free human serum albumin can be prepared by treatment of human serum albumin with charcoal at low pH. Likewise, treatment of human serum albumin with charcoal at low pH can be used to remove fatty acids from human serum albumin (Chen R F, J. Biol. Chem. 1967; 242:173-181).

Human serum albumin (HSA) is a highly soluble globular protein of Mr 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis andHemostasis, 6, 85-120, (1980)).

Human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)).

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

As used herein, the term “non-covalently bound complex” refers to a complex in which the bonds between the components of the complex are non-covalent bonds (e.g., weak bonds such as hydrogen bonds, electrostatic effects, n-effects, hydrophobic effects and Van der Waals forces). Further, human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Additionally, after the drug molecule binds to HSA, the drug molecule and HSA form a non-covalently bound drug and protein complex through the binding sites of HSA. This concept is commonly understood by one of ordinary skill in the art to which this disclosure belongs. One example of a non-covalently bound complex is a non-covalently bound complex of HSA and fatty acids, in which the fatty acids bind to HSA through HSA's multiple binding sites.

As used herein, the term “stable” refers to non-covalently bound complexes that do not readily disassociate and aggregate into their separate parts, e.g., do not readily dissociate and aggregate for a period of time of greater than 6 hours, 12 hours, 24 hours, or 3 days). For example, a solution including stable non-covalently bound complexes will often appear transparent whereas a solution including unstable non-covalently bound complexes will appear translucent or cloudy. Further, it will be appreciated by those of ordinary skill in the art, that after a period of time, stable non-covalently bound complexes can disassociate and aggregate into their separate parts. Thus, a solution including stable non-covalently bound complexes can become translucent or cloudy after a period of time (e.g., 6 hours, 12 hours, 24 hours, or 3 days).

Rolapitant is highly protein bound to human plasma (99.8%). The apparent volume of distribution (Vd/F) is 460 L in healthy subjects, indicating an extensive tissue distribution of rolapitant. In a population pharmacokinetic analysis of rolapitant, the Vd/F is 387 L in cancer patients. See VARUBI Prescribing Information.

As used herein, the term “essentially fatty acid free” refers to proteins (e.g. serum albumin) that contain less than about 0.02% fatty acid by weight. For example, human serum albumin that is essentially fatty acid free can contain less than 0.02% fatty acid by weight.

As used herein, the term “fatty acids” refers to non-esterified fatty acids (e.g. linoleic acid, α-linoleic acid, γ-linoleic acid).

As used herein the term rolapitant is a compound that has the CAS No. 552292-08-7 and the following chemical structure:

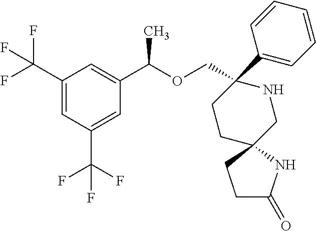 [SMILES: C[C@@H](OC[C@@]1(c2ccccc2)CC[C@]2(CCC(=O)N2)CN1)c1cc(C(F)(F)F)cc(C(F)(F)F)c1]

As used herein the term rolapitant hydrochloride is a compound that has the CAS No. 914462-92-3-7 and the following chemical structure:

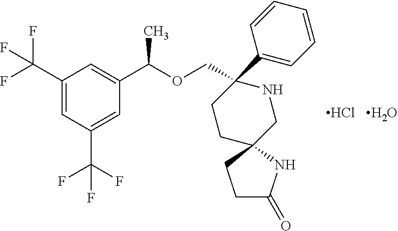 [SMILES: C[C@@H](OC[C@@]1(c2ccccc2)CC[C@]2(CCC(=O)N2)CN1)c1cc(C(F)(F)F)cc(C(F)(F)F)c1 ; Cl ; O]

Rolapitant hydrochloride is a white to off-white powder, with a molecular weight of 554.95. Solubility of rolapitant hydrochloride in aqueous solution is pH-dependent and is more soluble at lower pH.

Rolapitant has very low solubility at the physiological pH of 7.4 (<4 mcg/ml).

Rolapitant is indicated in combination with other antiemetic agents in adults for the prevention of delayed nausea and vomiting associated with initial and repeat courses of emetogenic cancer chemotherapy, including, but not limited to, highly emetogenic chemotherapy.

In some embodiments, rolapitant can be a pharmaceutically acceptable salt of rolapitant.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate (e.g., S-malate), tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, ethanedisulfonate, hydrogen bisulfide, bitartrate, gluconate, glucuronate, para-bromophenylsulfonate, carbonate, pyrosulfate, sulfite, bisulfite, monohydrogen phosphate, dihydrogen phosphate, metaphosphate, pyrophosphate, chloride, bromide, iodide, decanoate, caprylate, caprate, propiolate, suberate, sebacate, butyne-1,4-dioate, hexyne-1,6-dioate, terephthalate, sulfonate, xylenesulfonate, phenylpropionate, phenylbutyrate, β-hydroxybutyrate, glycolate, propanesulfonate, naphthalene-1-sulfonate, naphthalene-2-sulfonate and 2,5-dihydroxybenzoate. Suitable bases include pharmaceutically acceptable inorganic bases and pharmaceutically acceptable organic bases. Representative pharmaceutically acceptable base addition salts include hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, rolapitant is rolapitant hydrochloride.

In some embodiments, rolapitant is crystalline. In some embodiments, the rolapitant hydrochloride is crystalline. In some embodiments, the rolapitant hydrochloride is any one of the crystalline forms disclosed, for example, in U.S. Pat. Nos. 7,563,801, 7,981,905, 8,178,550, 8,470,842, 8,404,702, 9,345,692, and PCT application publication No. WO 2007/114922, the disclosures of each of the above are incorporated herein by reference in their entirety.

In some embodiments, rolapitant is in amorphous. In some embodiments. rolapitant is any one of the amorphous forms disclosed. In some embodiments, rolapitant hydrochloride is in amorphous. In some embodiments. rolapitant hydrochloride is any one of the amorphous forms disclosed.

In some embodiments, the composition is a clear aqueous solution when the composition is dissolved in an aqueous solvent. In some embodiments, the aqueous solution is substantially free of solvent other than water. In some embodiments, the aqueous solution is free of solvent other than water.

In some embodiments, the composition is a clear aqueous solution when the composition is dissolved in water. In some embodiments, the composition is a clear aqueous solution when the composition is dissolved in 0.9% saline. In some embodiments, the composition is a clear aqueous solution when the composition is dissolved in 5% Dextrose solution.

As used herein, the term “aqueous solution” refers to a solution, wherein at least one solvent is water and the weight % of water in the mixture of solvents is at least 50%, at least 60%, at least 70% or at least 90%. In some embodiments, aqueous solution is a solution in which water is the only solvent. In some embodiments, aqueous solution is 0.9% saline. The pH of the saline solution, for example, is about 5.4. In some embodiments, aqueous solution is 5% Dextrose solution. The pH of the saline solution, for example, is about 4.4. In some embodiments, aqueous solution is a buffer (e.g., phosphate buffer or a carbonate buffer). In some embodiments, the buffer is physiological buffer or a pharmaceutically acceptable buffer. In some embodiments, the buffer is any one of buffers described, for example, in Y.-C. Lee et al. International Journal of Pharmaceutics 253 (2003) 111-119, the disclosure of which is incorporated herein by reference in its entirety. In some embodiments, the buffer comprises maleic acid, tartaric acid, lactic acid, citric acid, acetic acid, sodium bicarbonate, sodium phosphate, or mixtures thereof. In some embodiments, the pH range of the buffer is from about 3 to about 9, from about 4 to about 8, from about 5 to about 7, from about 6.0 to about 6.5, from about 6 to about 7, from about 3 to about 5, from about 3 to about 7, from about 4 to about 6, or from about 5 to about 6. In some embodiments, the pH of the buffer is about 4, about 5, about 6, about 6.1, about 6.2, about 6.3, about 6.4, about 6.5, about 6.6, about 6.7, about 6.8, about 6.9, about 7.0, about 7.5, or about 8.

As used herein, the term “aqueous solvent” refer to a liquid comprising at least 50%, at least 60%, at least 70%, at least 90% or at least 95% water. In some embodiments, aqueous solvent is water, 0.9% saline or 5% dextrose.

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent.

In some embodiments, the composition forms a clear aqueous solution, when the composition is dissolved in an aqueous solvent, wherein the clear aqueous solution has pH value from about 5 to about 8. In some embodiments, the composition forms a clear aqueous solution, when the composition is dissolved in an aqueous solvent, wherein the clear aqueous solution has pH value from about 5.5 to about 7.8. In some embodiments, the composition forms a clear aqueous solution, when the composition is dissolved in an aqueous solvent, wherein the clear aqueous solution has pH value from about 6 to about 7.5. In some embodiments, the composition forms a clear aqueous solution, when the composition is dissolved in an aqueous solvent, wherein the clear aqueous solution has pH value from about 6.5 to about 7.5. In some embodiments, the composition forms a clear aqueous solution, when the composition is dissolved in an aqueous solvent, wherein the clear aqueous solution has pH value from about 6 to about 6.5. In some embodiments, the composition forms a clear aqueous solution, when the composition is dissolved in an aqueous solvent, wherein the clear aqueous solution has pH value from about 6.5 to about 7. In some embodiments, the composition forms a clear aqueous solution, when the composition is dissolved in an aqueous solvent, wherein the clear aqueous solution has pH value from about 7 to about 7.5. In some embodiments, the composition forms a clear aqueous solution, when the composition is dissolved in an aqueous solvent, wherein the clear aqueous solution has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous solution is substantially free of solvent other than water. In some embodiments, the aqueous solution is free of solvent other than water.

In some embodiments, the composition forms a clear aqueous solution, when the composition is dissolved in water, wherein the clear aqueous solution has pH value from about 5 to about 8. In some embodiments, the composition forms a clear aqueous solution, when the composition is dissolved in water, wherein the clear aqueous solution has pH value from about 5.5 to about 7.8. In some embodiments, the composition forms a clear aqueous solution, when the composition is dissolved in water, wherein the clear aqueous solution has pH value from about 6 to about 7.5. In some embodiments, the composition forms a clear aqueous solution, when the composition is dissolved in water, wherein the clear aqueous solution has pH value from about 6.5 to about 7.5. In some embodiments, the composition forms a clear aqueous solution, when the composition is dissolved in water, wherein the clear aqueous solution has pH value from about 6 to about 6.5. In some embodiments, the composition forms a clear aqueous solution, when the composition is dissolved in water, wherein the clear aqueous solution has pH value from about 6.5 to about 7. In some embodiments, the composition forms a clear aqueous solution, when the composition is dissolved in water, wherein the clear aqueous solution has pH value from about 7 to about 7.5. In some embodiments, the composition forms a clear aqueous solution, when the composition is dissolved in water, wherein the clear aqueous solution has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

In some embodiments, the term “clear aqueous solution” refers to an aqueous solution comprising rolapitant and HSA, that is transparent upon visual observation and essentially free of visible particles or precipitation of undissolved rolapitant. In some embodiments, “essentially free of visible particles or precipitation of undissolved rolapitant” can be assessed as follows: after a clear aqueous solution is filtered with a 0.22 micron filter, the amount of rolapitant in the filtered aqueous solution is at least 95% of the total amount of rolapitant in the aqueous solution before filtration. The total amount of rolapitant in the aqueous solution before filtration includes the particles or precipitation of undissolved rolapitant in the aqueous solution or with the aqueous solution. The amount of the rolapitant in an aqueous solution can be measured by the methods using HPLC. The methods of measuring the amount of the rolapitant in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

In some embodiments, when the composition comprising a non-covalently bound complex comprising rolapitant and human serum albumin as described herein (e.g., sterile solid powder) is dissolved in an aqueous solvent (e.g., water, 0.9% saline or 5% dextrose), the resultant aqueous solution, when filtered using a 0.22 micron filter, comprises at least 95% at the time of preparation, at least 95% after 1 hour, at least 95% after 2 hours, at least 95% after 3 hours, at least 95% after 4 hours, at least 95% after 5 hours, or at least 95% after 6 hours of the amount of rolapitant used to prepare the composition.

In some embodiments, when the composition comprising a non-covalently bound complex comprising rolapitant and human serum albumin as described herein (e.g., sterile solid powder) is dissolved in an aqueous solvent (e.g., water, 0.9% saline or 5% dextrose), the resultant aqueous solution, when filtered using a 0.22 micron filter, comprises at least 96% at the time of preparation, at least 96% after 1 hour, at least 96% after 2 hours, at least 96% after 3 hours, at least 96% after 4 hours, at least 96% after 5 hours, or at least 96% after 6 hours of the amount of rolapitant used to prepare the composition.

In some embodiments, the composition is a clear aqueous solution for at least about 1 hour, 2 hours, 3 hours, 4 hours, 5 hours, 6 hours, 8 hours, or 24 hours, when the composition is dissolved in an aqueous solvent. In some embodiments, the composition is a clear aqueous solution for at least about 1 hour, 2 hours, 3 hours, 4 hours, 5 hours, 6 hours, 8 hours, or 24 hours, when the composition is dissolved in water. In some embodiments, the composition is a clear aqueous solution for at least about 1 hour, 2 hours, 3 hours, 4 hours, 5 hours, 6 hours, 8 hours, or 24 hours, when the composition is dissolved in 0.9% saline. In some embodiments, the composition is a clear aqueous solution for at least about 1 hour, 2 hours, 3 hours, 4 hours, 5 hours, 6 hours, 8 hours, or 24 hours, when the composition is dissolved in 5% Dextrose solution.

In some embodiments, the amount of rolapitant that is bound to the HSA (e.g., non-covalently) in the aqueous solution (e.g., clear aqueous solution) comprising the composition comprising a non-covalently bound complex comprising rolapitant and human serum albumin (as described herein) is at least 40%, at least 50%, at least 60%, at least 70%, at least 80%, at least 90%, at least 95%, at least 99%, or about 100% of the total about of rolapitant in the aqueous solution.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

In some embodiments, the aqueous formulation is a clear aqueous solution reconstituted from the solid formulation (e.g. the sterile lyophilized powder) in water. In some embodiments, the aqueous formulation is a clear aqueous solution reconstituted from the solid formulation (e.g. the sterile lyophilized powder) in 0.9% saline. In some embodiments, the aqueous formulation is a clear aqueous solution reconstituted from the solid formulation (e.g. the sterile lyophilized powder) in 5% Dextrose solution.

In some embodiments, the aqueous formulation has pH value from about 5 to about 8. In some embodiments, the aqueous formulation has pH value from about 5.5 to about 7.8. In some embodiments, the aqueous formulation has pH value from about 6 to about 7.5. In some embodiments, the aqueous formulation has pH value from about 6.5 to about 7.5. In some embodiments, the aqueous formulation has pH value from about 6 to about 6.5. In some embodiments, the aqueous formulation has pH value from about 6.5 to about 7. In some embodiments, the aqueous formulation has pH value from about 7 to about 7.5. In some embodiments, the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution, wherein the aqueous formulation has pH value from about 5 to about 8, and wherein the aqueous formulation is substantially free of solvent other than water. In some embodiments, the aqueous formulation is a clear aqueous solution, wherein the aqueous formulation has pH value from about 5 to about 8, and wherein the aqueous formulation is free of solvent other than water. In some embodiments, the aqueous formulation is a clear aqueous solution, wherein the aqueous formulation has pH value from about 5.5 to about 7.8, and wherein the aqueous formulation is substantially free of solvent other than water. In some embodiments, the aqueous formulation is a clear aqueous solution, wherein the aqueous formulation has pH value from about 5.5 to about 7.8, and wherein the aqueous formulation is free of solvent other than water. In some embodiments, the aqueous formulation is a clear aqueous solution, wherein the aqueous formulation has pH value from about 6 to about 7.5, and wherein the aqueous formulation is substantially free of solvent other than water. In some embodiments, the aqueous formulation is a clear aqueous solution, wherein the aqueous formulation has pH value from about 6 to about 7.5, and wherein the aqueous formulation is free of solvent other than water. In some embodiments, the aqueous formulation is a clear aqueous solution, wherein the aqueous formulation has pH value from about 6.5 to about 7.5, and wherein the aqueous formulation is substantially free of solvent other than water. In some embodiments, the aqueous formulation is a clear aqueous solution, wherein the aqueous formulation has pH value from about 6.5 to about 7.5, and wherein the aqueous formulation is free of solvent other than water. In some embodiments, the aqueous formulation is a clear aqueous solution, wherein the aqueous formulation has pH value from about 6 to about 6.5, and wherein the aqueous formulation is substantially free of solvent other than water. In some embodiments, the aqueous formulation is a clear aqueous solution, wherein the aqueous formulation has pH value from about 6 to about 6.5, and wherein the aqueous formulation is free of solvent other than water. In some embodiments, the aqueous formulation is a clear aqueous solution, wherein the aqueous formulation has pH value from about 6.5 to about 7, and wherein the aqueous formulation is substantially free of solvent other than water. In some embodiments, the aqueous formulation is a clear aqueous solution, wherein the aqueous formulation has pH value from about 6.5 to about 7, and wherein the aqueous formulation is free of solvent other than water. In some embodiments, the aqueous formulation is a clear aqueous solution, wherein the aqueous formulation has pH value from about 7 to about 7.5, and wherein the aqueous formulation is substantially free of solvent other than water. In some embodiments, the aqueous formulation is a clear aqueous solution, wherein the aqueous formulation has pH value from about 7 to about 7.5, and wherein the aqueous formulation is free of solvent other than water. In some embodiments, the aqueous formulation is a clear aqueous solution, wherein the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5, and wherein the aqueous formulation is substantially free of solvent other than water. In some embodiments, the aqueous formulation is a clear aqueous solution, wherein the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5, and wherein the aqueous formulation is free of solvent other than water.

In some embodiments, the aqueous formulation is a clear aqueous solution for at least 1 hour, 2 hours, 3 hours, 4 hours, 5 hours, 6 hours, 8 hours, or 24 hours. In some embodiments, the solution remains clear for at least about 2 hours, 4 hours, 6 hours, 8 hours, 10 hours, 12 hours, 20 hours, 24 hours, 2 days, 3 days, 4 days, 5 days, 6 days or a week. In some embodiments, the aqueous formulation is substantially free of solvent other than water. In some embodiments, the aqueous formulation is free of solvent other than water.

In some embodiments, after the aqueous formulation (e.g. a clear aqueous solution) is filtered by a 0.22 micron filter, the amount of rolapitant in the filtered aqueous solution is at least 95% of the total amount of rolapitant in the aqueous solution before the filtration. In some embodiments, after the aqueous formulation (e.g. a clear aqueous solution) is filtered by a 0.22 micron filter, the amount of rolapitant in the filtered aqueous solution is at least 96% of the total amount of rolapitant in the aqueous solution before the filtration. In some embodiments, after the aqueous formulation (e.g. a clear aqueous solution) is filtered by a 0.22 micron filter, the amount of rolapitant in the filtered aqueous solution is at least 97% of the total amount of rolapitant in the aqueous solution before the filtration. In some embodiments, after the aqueous formulation (e.g. a clear aqueous solution) is filtered by a 0.22 micron filter, the amount of rolapitant in the filtered aqueous solution is at least 98% of the total amount of rolapitant in the aqueous solution before the filtration. In some embodiments, after the aqueous formulation (e.g. a clear aqueous solution) is filtered by a 0.22 micron filter, the amount of rolapitant in the filtered aqueous solution is at least 99% of the total amount of rolapitant in the aqueous solution before the filtration. In some embodiments, after the aqueous formulation (e.g. a clear aqueous solution) is filtered by a 0.22 micron filter, the amount of rolapitant in the filtered aqueous solution is at least 99.5% of the total amount of rolapitant in the aqueous solution before the filtration. In some aspects of these embodiments, the aqueous formulation is filtered by a 0.22-micron filter at a time period selected from 0 hour, 1 hour, 2 hours, 3, hours, 4 hours, 5 hours, 6 hours, 12 hours, 18 hours and 24 hours. In some embodiments, the aqueous formulation is free of solvent other than water. In some embodiments, the aqueous formulation is substantially free of solvent other than water.

In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 5 to about 8, and wherein the aqueous formulation is substantially free of solvent other than water. In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 6 to about 7.5, and wherein the aqueous formulation is substantially free of solvent other than water. In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 5 to about 8, and wherein the aqueous formulation is free of solvent other than water. In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 6 to about 7.5, and wherein the aqueous formulation is free of solvent other than water. In some aspects of these embodiments, the aqueous formulation is filtered by a 0.22-micron filter at a time period selected from 0 hour, 1 hour, 2 hours, 3, hours, 4 hours, 5 hours, 6 hours, 8 hours, 12 hours, 18 hours and 24 hours.

Also, provided herein is a pharmaceutical composition comprising the composition comprising a non-covalently bound complex comprising the rolapitant and the human serum albumin as described herein, and a pharmaceutically acceptable carrier.

In some embodiments, the pharmaceutical composition comprising the composition comprising a non-covalently bound complex comprising the rolapitant and the human serum albumin as described herein, and a pharmaceutically acceptable carrier, is a liquid pharmaceutical composition. In some embodiments, the liquid pharmaceutical composition is an aqueous solution. In some embodiments, the liquid pharmaceutical composition is free of solvent other than water. In some embodiments, the liquid pharmaceutical composition is substantially free of solvent other than water. In some embodiments, the liquid pharmaceutical composition is an injectable pharmaceutical formulation. In some embodiments, the liquid pharmaceutical composition is a formulation for infusion (e.g., intravenous infusion).

In some embodiments, the pharmaceutically acceptable carrier refers to any carrier useful to solubilize and deliver an agent to a subject. A desirable pharmaceutically acceptable carrier is saline. Other pharmaceutically acceptable carrier and their formulation are known to one skilled in the art and described, for example, in Remington's Pharmaceutical Sciences. (20^{th} edition), ed. A. Gennaro, 2003, Lippincon Williams & Wilkins. In some embodiments, the carrier may contain components such as, for example, dextrose, glucose, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, and cellulose-based substances. In some embodiments, the carrier may contain components such as contain anti-oxidants, buffers, bacteriostats and solutes which render the formulation isotonic with the blood of the intended recipient.

In some embodiments, the pharmaceutically acceptable excipient is selected from lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical compositions may additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The pharmaceutical composition may be formulated so as to provide quick, sustained or delayed release of the active ingredient after administration to the patient by employing procedures known in the art.

In some embodiments, the pharmaceutical composition of the present disclosure may be administered by a syringe or a catheter, or any other means generally known in the art for the delivery of a pharmaceutical agent by injection to the subject in need thereof. The delivery means will vary, as recognized by those skilled in the art, depending on the diseases and conditions treated, the severity of the disease, the sex, age and general health condition of the subject, excipient usage, the possibility of co-usage with other therapeutic treatments such as use of other agents as described herein and the judgment of the treating physician.

The pharmaceutical composition comprising the composition comprising a non-covalently bound complex comprising the rolapitant and the human serum albumin as described herein can be administered to a subject via various routes, such as parenterally, including intravenous, intra-arterial, intraperitoneal, intrapulmonary, oral, inhalation, intravascular, intramuscular, intra-tracheal, subcutaneous, intraocular, intrathecal, or transdermal. For example, the composition can be administered by inhalation to treat conditions of the respiratory tract. In some embodiments, the pharmaceutical composition is administrated in a unit dosage form such as capsule or oral suspension (e.g., for pediatric patients 6 months to less than 12 years of age).

In some embodiments, the pharmaceutical composition is free of a vehicle selected from the group consisting of water soluble organic solvents, non-ionic surfactants, water insoluble lipids, organic lipids/semisolids and phospholipids. Water soluble organic solvents may be selected from, for example, polyethylene glycol 300, polyethylene glycol 400, ethanol, propylene glycol, glycerin, N-methyl-2-pyrrolidone, dimethylacetamide and dimethylsulfoxide. Non-ionic surfactants may be selected from Cremophor EL, Cremophor RH 40, Cremophor RH 60, d-a-tocopherol polyethylene glycol 1000 succinate, polysorbate 80, Solutol HS 15, sorbitan monooleate, poloxamer 407, Labrifil M-1944CS, Labrafil M-2125CS, Labrasol, Gellucire 44/14, Softigen 767, and mono- and di-fatty acid esters of PEG 300, 400 or 1750. The water insoluble lipids are selected from castor oil, corn oil, cottonseed oil, olive oil, peanut oil, peppermint oil, safflower oil, sesame oil, soybean oil, hydrogenated vegetable oils, hydrogenated soybean oil, and medium chain triglycerides of coconut oil and palm seed oil. Organic liquids and semisolids may be selected from beeswax, d-a-tocopherol, oleic acid and medium chain mono- and diglycerides. The phospholipids may be selected from lecithin, hydrogenated soy phosphatidylcholine, distearoylphosphatidylglycerol, L-a-dimyri stoylphosphatidylcholine and L-a-dimyristoylphosphatidylglycerol.

In some embodiments, the pharmaceutical composition is free of macrogol 15-hydroxystearate. In some embodiments, the pharmaceutical composition is free of a medium chain triglyceride. In some embodiments, the pharmaceutical composition is free of a long chain triglyceride. In some embodiments, the pharmaceutical composition is free of soybean oil. In some embodiments, the pharmaceutical composition can be substantially free of a vehicle selected from the group consisting of macrogol 15-hydroxystearate, a medium chain triglyceride, a long chain triglyceride, and soybean oil. In some embodiments, the pharmaceutical composition can be free of a vehicle selected from the group consisting of macrogol 15-hydroxystearate, a medium chain triglyceride, a long chain triglyceride, and soybean oil.

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method for the prevention of chemotherapy-induced nausea and vomiting (CINV) or postoperative nausea and vomiting (PONV), the method comprising the step of administering to a subject in need thereof of a therapeutically effective amount of a pharmaceutical composition comprising the composition comprising a non-covalently bound complex comprising the rolapitant and the human serum albumin as described herein, and a pharmaceutically acceptable carrier. In some embodiments, the methods described herein are for the prevention of chemotherapy-induced nausea and vomiting (CINV). In some embodiments, the methods described herein are for the prevention of delayed nausea and vomiting associated with initial and repeat courses of emetogenic cancer chemotherapy. Including, but not limited to, highly emetogenic cancer chemotherapy (HEC). In some embodiments, the methods described herein are for the prevention of nausea and vomiting associated with initial and repeat courses of highly emetogenic cancer chemotherapy (HEC). In some embodiments, the methods described herein are for the prevention of nausea and vomiting associated with initial and repeat courses of moderately emetogenic cancer chemotherapy (MEC). In some embodiments, the methods described herein are for the prevention of postoperative nausea and vomiting (PONV). In some embodiments, the methods described herein are for the prevention of cough (e.g., chronic cough from unknown cause).

As used herein, the term “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, prevention of a disease, condition or disorder refers to decreasing the possibility of acquiring the disease, condition or disorder and/or its associated symptoms. In some embodiments, reducing the risk of a disease, condition or disorder refers to completely or almost completely stopping the disease, condition or disorder from occurring.

As used herein, the terms “individual”, “patient”, or “subject” are used interchangeably and refer to any animal, including mammals, preferably mice, rats, other rodents, rabbits, dogs, cats, swine, cattle, sheep, horses, or primates, and most preferably humans.

As used herein, an “effective amount,” “therapeutically effective amount,” or a “pharmaceutically-effective amount” in reference to the compounds or compositions of the instant invention refers to the amount sufficient to induce a desired biological, pharmacological, or therapeutic outcome in a subject. That result can be reduction, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In cancer treatment, the result will generally include the reduction, mitigation, limitation, and/or, delay of the deleterious physiological manifestations, growth or metastases of neoplasms.

Also, provided herein is a method of treating chemotherapy-induced nausea and vomiting, the method comprising the step of administering to a subject in need thereof of a therapeutically effective amount of a pharmaceutical composition comprising the composition comprising a non-covalently bound complex comprising the rolapitant and the human serum albumin as described herein, and a pharmaceutically acceptable carrier. In some aspects of these embodiments, the chemotherapy-induced nausea and vomiting is delayed nausea and vomiting associated with initial and repeat courses of emetogenic cancer chemotherapy. Including, but not limited to, highly emetogenic cancer chemotherapy (HEC). In other aspects of these embodiments, the chemotherapy-induced nausea and vomiting is nausea and vomiting associated with initial and repeat courses of moderately emetogenic cancer chemotherapy (MEC). In other aspects of these embodiments, the chemotherapy-induced nausea and vomiting is nausea and vomiting associated with initial and repeat courses of highly emetogenic cancer chemotherapy (HEC). In some embodiments, the methods described herein are for the treating cough (e.g., chronic cough from unknown cause).

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

In some embodiments, the methods described herein are performed in combination with at least one other antiemetic agents. In some embodiments, the methods described herein are performed in combination with dexamethasone. In some embodiments, the methods described herein are performed in combination with dexamethasone and a 5-HT3 antagonist (e.g., tropisetron, palonosetron, ramosetron, granisetron, ondansetron, dolasetron, or metoclopramide). In some embodiments, the antiemetic agent is selected from tropisetron, palonosetron, ramosetron, granisetron, ondansetron, dolasetron, metoclopramide, domperidone, olanzapine, droperidol, haloperidol, chlorpromazine, prochlorperazine, alizapride, prochlorperazine, metoclopramide, casopitant, cyclizine, diphenhydramine, dimenhydrinate, doxylamine, meclizine, promethazine, hydroxyzine, dronabinol, sativex, midazolam, lorazepam, hyoscine, trimethobenzamide, emetrol, propofol and muscimol.

In some embodiments, provided herein is a method for preventing chemotherapy-induced nausea and vomiting (e.g., acute and delayed nausea and vomiting associated with initial and repeat courses of emetogenic cancer chemotherapy. Including, but not limited to, highly emetogenic cancer chemotherapy (HEC); or of delayed nausea and vomiting associated with initial and repeat courses of highly emetogenic cancer chemotherapy (HEC), or of delayed nausea and vomiting associated with initial and repeat courses of highly emetogenic cancer chemotherapy (HEC), or postoperative nausea and vomiting (PONV) in adults), the method comprising administering to a subject in need thereof a therapeutically effective amount a pharmaceutical composition comprising the composition comprising the rolapitant and the human serum albumin as described herein, and a therapeutically effective amount of at least one antiemetic agent as described herein.

As used herein, the term “emetogenic chemotherapy” refers to chemotherapy during which a patient experiences emesis in the absence of effective antiemetic prophylaxis. Level 1 emetogenic chemotherapy refers to chemotherapy in which less than 10% of patients experience emesis; level 2 emetogenic chemotherapy refers to chemotherapy in which 10-30% of patients experience emesis; level 3 emetogenic chemotherapy refers to chemotherapy in which 30-60% of patients experience emesis; level 4 emetogenic chemotherapy refers to chemotherapy in which 60-90% of patients experience emesis; and level 5 emetogenic chemotherapy refers to chemotherapy in which more than 90% of patients experience emesis in the absence of prophylaxis. In some embodiments, level 3-5 cancer chemotherapy highly emetogenic (HEC). In some embodiments, level 1-2 cancer chemotherapy moderately emetogenic (MEC).

In some embodiments, emetogenic chemotherapy is cisplatin-based or taxane-based chemotherapy. In some embodiments, emetogenic chemotherapy is a therapy with an anti-cancer agent selected from carmustine, cisplatin, cyclophosphamide, dacarbazine, mechlorethamine, streptozocin, carboplatin, cytarabine, doxorubicin, methotrexate, procarbazine, cyclophosphamid, epirubicin, hexamethylmelamine, idarubicin, ifosfamide, irinotecan, mitoxantrone, capecitabine, docetaxel, etoposide, 5-fluorouracil, gemcitabine, mitomycin, paclitaxel, topotecan, bleomycin, busulfan, chlorambucil, 2-chlorodeoxyadenosine, fludarabine, hydroxyurea, L-phenylalanine mustard, thioguanine, vinblastine, vincristine and vinorelbine.

In some embodiments, a pharmaceutical composition comprising the composition comprising a non-covalently bound complex comprising the rolapitant and the human serum albumin as described herein and an antiemetic agent are administered simultaneously.

In some embodiments, a pharmaceutical composition comprising the composition comprising a non-covalently bound complex comprising the rolapitant and the human serum albumin as described herein and an antiemetic agent are administered consecutively.

Also, provided herein is a pharmaceutical composition comprising the composition comprising a non-covalently bound complex comprising the rolapitant and the human serum albumin as described herein, at least one antiemetic agent as described herein, and a pharmaceutically acceptable carrier.

In some embodiments, provided herein is a pharmaceutical composition comprising the composition comprising a non-covalently bound complex comprising the rolapitant and the human serum albumin as described herein, a 5-HT3 antagonist (e.g., tropisetron, palonosetron, ramosetron, granisetron, ondansetron, dolasetron, or metoclopramide), and a pharmaceutically acceptable carrier.

In some embodiments, provided herein is a pharmaceutical composition comprising the composition comprising a non-covalently bound complex comprising the rolapitant and the human serum albumin as described herein, palonosetron, and a pharmaceutically acceptable carrier.

In some embodiments, provided herein is a pharmaceutical composition comprising the composition comprising a non-covalently bound complex comprising the rolapitant and the human serum albumin as described herein, 0.5 mg of palonosetron, and a pharmaceutically acceptable carrier.

In some embodiments, provided herein is a pharmaceutical composition comprising the composition comprising a non-covalently bound complex comprising the rolapitant and the human serum albumin as described herein, 0.25 mg of palonosetron, and a pharmaceutically acceptable carrier.

In some embodiments, provided herein is a pharmaceutical composition comprising the composition comprising a non-covalently bound complex comprising the rolapitant and the human serum albumin as described herein, 0.28 mg of palonosetron hydrochloride, and a pharmaceutically acceptable carrier.

In some embodiments, provided herein is a pharmaceutical composition comprising the composition comprising a non-covalently bound complex comprising the rolapitant and the human serum albumin as described herein, about 0.25 mg of palonosetron, and a pharmaceutically acceptable carrier.

In some embodiments, provided herein is a pharmaceutical composition comprising the composition comprising a non-covalently bound complex comprising the rolapitant and the human serum albumin as described herein, about 0.28 mg of palonosetron hydrochloride, and a pharmaceutically acceptable carrier.

In some embodiments, provided herein is a liquid pharmaceutical formulation for injection comprising the composition comprising a non-covalently bound complex comprising the rolapitant and the human serum albumin as described herein, 0.25 mg of palonosetron, and a pharmaceutically acceptable carrier.

In some embodiments, provided herein is a liquid pharmaceutical formulation for injection comprising the composition comprising a non-covalently bound complex comprising the rolapitant and the human serum albumin as described herein, 0.28 mg of palonosetron hydrochloride, and a pharmaceutically acceptable carrier.

In some embodiments, the liquid pharmaceutical formulation for injection is an aqueous solution. In some embodiments, the liquid pharmaceutical formulation for injection is free of solvent other than water. In some embodiments, the liquid pharmaceutical formulation for injection is substantially free of solvent other than water. In some embodiments, the liquid pharmaceutical formulation for injection is a formulation for infusion (e.g., intravenous infusion).

In some embodiments, the liquid pharmaceutical formulation for injection has pH value from about 5 to about 8. In some embodiments, the liquid pharmaceutical formulation for injection has pH value from about 5.5 to about 7.8. In some embodiments, the liquid pharmaceutical formulation for injection has pH value from about 6 to about 7.5. In some embodiments, the liquid pharmaceutical formulation for injection has pH value from about 6.5 to about 7.5. In some embodiments, the liquid pharmaceutical formulation for injection has pH value from about 6 to about 6.5. In some embodiments, the liquid pharmaceutical formulation for injection has pH value from about 6.5 to about 7. In some embodiments, the liquid pharmaceutical formulation for injection has pH value from about 7 to about 7.5. In some embodiments, the liquid pharmaceutical formulation for injection has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the liquid pharmaceutical formulation for injection is substantially free of solvent other than water. In some embodiments, the liquid pharmaceutical formulation for injection is free of solvent other than water.

As used herein the term “palonosetron” is a compound that has the CAS No. 135729-61-2 and the following chemical structure:

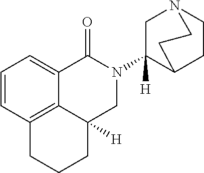 [SMILES: O=C1c2cccc3c2[C@H](CCC3)CN1[C@@H]1CN2CCC1CC2]

In some embodiments, the palonosetron can be a pharmaceutically acceptable salt of palonosetron. In some embodiments, the palonosetron can be palonosetron hydrochloride.

As used herein the term “palonosetron hydrochloride” is a compound that has the CAS No. 135729-62-3 and the following chemical structure:

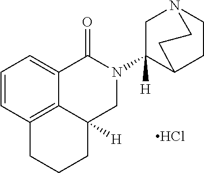 [SMILES: O=C1c2cccc3c2[C@H](CCC3)CN1[C@@H]1CN2CCC1CC2 ; Cl]

In some embodiments, provided herein is a pharmaceutical composition comprising the composition comprising a non-covalently bound complex comprising the rolapitant and the human serum albumin as described herein, granisetron, and a pharmaceutically acceptable carrier.

In some embodiments, provided herein is a pharmaceutical composition comprising the composition comprising a non-covalently bound complex comprising the rolapitant and the human serum albumin as described herein, ondansetron, and a pharmaceutically acceptable carrier.

The methods described herein may be performed alone or in conjunction with another therapy, such as surgery, radiation, chemotherapy, immunotherapy, gene therapy, and the like. In some embodiments, a composition comprising the rolapitant and the human serum albumin as described herein is administered prior to chemotherapy.

In some embodiments, the amount of rolapitant that is administered to a subject in need thereof with any one of pharmaceutical compositions described herein is from about 50 mg to about 250 mg, from about 60 mg to about 240 mg, from about 70 mg to 230 mg, from about 80 mg to about 220 mg, from about 90 mg to about 210 mg, from about 100 mg to about 200 mg, or from about 100 mg to about 180 mg. In some embodiments, the amount of rolapitant that is administered to a subject in need thereof with any one of pharmaceutical compositions described herein is about 100 mg, about 125 mg, about 150 mg, about 160 mg, about 165 mg, about 170 mg, about 175, about 180 mg, about 185 mg, about 190 mg, about 200 mg, about 210 mg, about 220 mg, about 225 mg, or about 250 mg.

In some embodiments, a composition comprising the rolapitant and the human serum albumin as described herein is administered IV (intravenously). In some embodiments, a composition comprising the rolapitant and the human serum albumin as described herein is administered as an infusion in a dosage from about 0.1 mg/ml to about 20 mg/ml, from about 0.2 mg/ml to about 15 mg/ml, from about 0.5 mg/ml to about 12 mg/ml, from about 0.6 mg/ml to about 10 mg/ml, from about 0.7 mg/ml to about 8 mg/ml, from about 0.8 mg/ml to about 6 mg/ml, from about 0.9 mg/ml to about 5 mg/ml, or from about 1 mg/ml to about 3 mg/ml. In some embodiments, a composition comprising the rolapitant and the human serum albumin as described herein is administered as an infusion in a dosage of about 0.2 mg/ml, 0.5 mg/ml, about 0.75 mg/ml, about 1 mg/ml, about 1.25 mg/ml, about 1.5 mg/ml, about 1.75 mg/ml, about 2 mg/ml, about 2.25 mg/ml, about 2.5 mg/ml, about 2.75 mg/ml, about 3 mg/ml, about 4 mg/ml, or about mg/ml. In some embodiments, a composition comprising the rolapitant and the human serum albumin as described herein is administered as an infusion for a time period from about 1 min to about 24 hours, from about 5 min to about 18 hours, from about 10 min to about 14 hours, from about 15 min to about 12 hours, from about 20 min to about 10 hours, from about 30 min to about 10 hours, or from about 1 hour to about 8 hours. In some embodiments, a composition comprising the rolapitant and the human serum albumin as described herein is administered as an infusion for a time period of about 10 min, about 15 min, about 30 min, about 45 min, about 1 hour, about 3 hours, about 5 hours, or about 10 hours.

As will be understood by those of ordinary skill in the art, the appropriate doses of rolapitant will be approximately those already employed in clinical therapies wherein rolapitant is administered alone or in combination with other therapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for rolapitant.

Also, provided herein is a composition comprising rolapitant and human serum albumin, wherein the rolapitant and the human serum albumin in the composition have a ratio by weight from about 1:20 to about 1:2000.

In some embodiments, the rolapitant and the human serum albumin in the composition have a ratio by weight from about 1:100 to about 1:1000, from about 1:130 to about 1:800, from about 1:140 to about 1:600, about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:175, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

In some embodiments, the rolapitant can be a pharmaceutically acceptable salt of rolapitant. In some embodiments, rolapitant is rolapitant hydrochloride. In some embodiments, rolapitant can be any one of crystal forms, amorphous forms, solvates and hydrates as described herein. In some embodiments, rolapitant hydrochloride can be any one of crystal forms, amorphous forms, solvates and hydrates as described herein.

In some embodiments, the composition is a clear aqueous solution when the composition is dissolved in an aqueous solvent. In some embodiments, the aqueous solution is substantially free of solvent other than water. In some embodiments, the aqueous solution is free of solvent other than water.

In some embodiments, the composition is a clear aqueous solution when the composition is dissolved in water. In some embodiments, the composition is a clear aqueous solution when the composition is dissolved in 0.9% saline. In some embodiments, the composition is a clear aqueous solution when the composition is dissolved in 5% Dextrose solution.

As used herein, the term “aqueous solution” refers to a solution, wherein at least one solvent is water and the weight % of water in the mixture of solvents is at least 50%, at least 60%, at least 70% or at least 90%. In some embodiments, aqueous solution is a solution in which water is the only solvent. In some embodiments, aqueous solution is 0.9% saline. The pH of the saline solution, for example, is about 5.4. In some embodiments, aqueous solution is 5% Dextrose solution. The pH of the 5% Dextrose solution, for example, is about 4.4. In some embodiments, aqueous solution is a buffer (e.g., phosphate buffer or a carbonate buffer). In some embodiments, the buffer is physiological buffer or a pharmaceutically acceptable buffer. In some embodiments, the buffer is any one of buffers described, for example, in Y.-C. Lee et al. International Journal of Pharmaceutics 253 (2003) 111-119, the disclosure of which is incorporated herein by reference in its entirety. In some embodiments, the buffer comprises maleic acid, tartaric acid, lactic acid, citric acid, acetic acid, sodium bicarbonate, sodium phosphate, or mixtures thereof. In some embodiments, the pH range of the buffer is from about 3 to about 9, from about 4 to about 8, from about 5 to about 7, from about 6.0 to about 6.5, from about 6 to about 7, from about 3 to about 5, from about 3 to about 7, from about 4 to about 6, or from about 5 to about 6. In some embodiments, the pH of the buffer is about 4, about 5, about 6, about 6.1, about 6.2, about 6.3, about 6.4, about 6.5, about 6.6, about 6.7, about 6.8, about 6.9, about 7.0, about 7.5, or about 8.

As used herein, the term “aqueous solvent” refer to a liquid comprising at least 50%, at least 60%, at least 70%, at least 90% or at least 95% water. In some embodiments, aqueous solvent is water, 0.9% saline or 5% dextrose.

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent.

As used herein, the term “clear aqueous solution” refers to an aqueous solution containing rolapitant and HSA that is transparent upon visual observation and essentially free of visible particles or precipitation of undissolved rolapitant.

The term “essentially free of visible particles or precipitation of undissolved rolapitant” can be assessed as follows: after a clear aqueous solution is filtered with a 0.22 micron filter, the amount of rolapitant in the filtered aqueous solution is at least 95% of the total amount of rolapitant in the aqueous solution before filtration. The total amount of rolapitant in the aqueous solution before filtration includes the particles or precipitation of undissolved rolapitant in the aqueous solution or with the aqueous solution. The amount of the rolapitant in an aqueous solution can be measured by the methods using HPLC. The methods of measuring the amount of the rolapitant in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter. In some embodiments, the term “micron” refers to a micrometer.

In some embodiments, the composition forms a clear aqueous solution, when the composition is dissolved in an aqueous solvent, wherein the clear aqueous solution has pH value from about 5 to about 8. In some embodiments, the composition forms a clear aqueous solution, when the composition is dissolved in an aqueous solvent, wherein the clear aqueous solution has pH value from about 5.5 to about 7.8. In some embodiments, the composition forms a clear aqueous solution, when the composition is dissolved in an aqueous solvent, wherein the clear aqueous solution has pH value from about 6 to about 7.5. In some embodiments, the composition forms a clear aqueous solution, when the composition is dissolved in an aqueous solvent, wherein the clear aqueous solution has pH value from about 6.5 to about 7.5. In some embodiments, the composition forms a clear aqueous solution, when the composition is dissolved in an aqueous solvent, wherein the clear aqueous solution has pH value from about 6 to about 6.5. In some embodiments, the composition forms a clear aqueous solution, when the composition is dissolved in an aqueous solvent, wherein the clear aqueous solution has pH value from about 6.5 to about 7. In some embodiments, the composition forms a clear aqueous solution, when the composition is dissolved in an aqueous solvent, wherein the clear aqueous solution has pH value from about 7 to about 7.5. In some embodiments, the composition forms a clear aqueous solution, when the composition is dissolved in an aqueous solvent, wherein the clear aqueous solution has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous solution is substantially free of solvent other than water. In some embodiments, the aqueous solution is free of solvent other than water.

In some embodiments, the composition forms a clear aqueous solution, when the composition is dissolved in water, wherein the clear aqueous solution has pH value from about 5 to about 8. In some embodiments, the composition forms a clear aqueous solution, when the composition is dissolved in water, wherein the clear aqueous solution has pH value from about 5.5 to about 7.8. In some embodiments, the composition forms a clear aqueous solution, when the composition is dissolved in water, wherein the clear aqueous solution has pH value from about 6 to about 7.5. In some embodiments, the composition forms a clear aqueous solution, when the composition is dissolved in water, wherein the clear aqueous solution has pH value from about 6.5 to about 7.5. In some embodiments, the composition forms a clear aqueous solution, when the composition is dissolved in water, wherein the clear aqueous solution has pH value from about 6 to about 6.5. In some embodiments, the composition forms a clear aqueous solution, when the composition is dissolved in water, wherein the clear aqueous solution has pH value from about 6.5 to about 7. In some embodiments, the composition forms a clear aqueous solution, when the composition is dissolved in water, wherein the clear aqueous solution has pH value from about 7 to about 7.5. In some embodiments, the composition forms a clear aqueous solution, when the composition is dissolved in water, wherein the clear aqueous solution has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

In some embodiments, the composition forms a clear aqueous solution, when the composition is dissolved in 0.9% saline, wherein the clear aqueous solution has pH value from about 5 to about 8. In some embodiments, the composition forms a clear aqueous solution, when the composition is dissolved in 0.9% saline, wherein the clear aqueous solution has pH value from about 5.5 to about 7.8. In some embodiments, the composition forms a clear aqueous solution, when the composition is dissolved in 0.9% saline, wherein the clear aqueous solution has pH value from about 6 to about 7.5. In some embodiments, the composition forms a clear aqueous solution, when the composition is dissolved in 0.9% saline, wherein the clear aqueous solution has pH value from about 6.5 to about 7.5. In some embodiments, the composition forms a clear aqueous solution, when the composition is dissolved in 0.9% saline, wherein the clear aqueous solution has pH value from about 6 to about 6.5. In some embodiments, the composition forms a clear aqueous solution, when the composition is dissolved in 0.9% saline, wherein the clear aqueous solution has pH value from about 6.5 to about 7. In some embodiments, the composition forms a clear aqueous solution, when the composition is dissolved in 0.9% saline, wherein the clear aqueous solution has pH value from about 7 to about 7.5. In some embodiments, the composition forms a clear aqueous solution, when the composition is dissolved in 0.9% saline, wherein the clear aqueous solution has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

In some embodiments, the composition forms a clear aqueous solution, when the composition is dissolved in 5% Dextrose solution, wherein the clear aqueous solution has pH value from about 5 to about 8. In some embodiments, the composition forms a clear aqueous solution, when the composition is dissolved in 5% Dextrose solution, wherein the clear aqueous solution has pH value from about 5.5 to about 7.8. In some embodiments, the composition forms a clear aqueous solution, when the composition is dissolved in 5% Dextrose solution, wherein the clear aqueous solution has pH value from about 6 to about 7.5. In some embodiments, the composition forms a clear aqueous solution, when the composition is dissolved in 5% Dextrose solution, wherein the clear aqueous solution has pH value from about 6.5 to about 7.5. In some embodiments, the composition forms a clear aqueous solution, when the composition is dissolved in 5% Dextrose solution, wherein the clear aqueous solution has pH value from about 6 to about 6.5. In some embodiments, the composition forms a clear aqueous solution, when the composition is dissolved in 5% Dextrose solution, wherein the clear aqueous solution has pH value from about 6.5 to about 7. In some embodiments, the composition forms a clear aqueous solution, when the composition is dissolved in 5% Dextrose solution, wherein the clear aqueous solution has pH value from about 7 to about 7.5. In some embodiments, the composition forms a clear aqueous solution, when the composition is dissolved in 5% Dextrose solution, wherein the clear aqueous solution has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5.

In some embodiments, when the composition comprising rolapitant and human serum albumin as described herein (e.g., sterile solid powder) is dissolved in an aqueous solvent (e.g., water, 0.9% saline or 5% dextrose), the resultant aqueous solution, when filtered using a 0.22 micron filter, comprises at least 95% at the time of preparation, at least 95% after 1 hour, at least 95% after 2 hours, at least 95% after 3 hours, at least 95% after 4 hours, at least 95% after 5 hours, or at least 95% after 6 hours of the amount of rolapitant used to prepare the composition.

In some embodiments, the composition is a clear aqueous solution when the composition is dissolved in an aqueous solvent (e.g. water, 0.9% saline solution, or 5% dextrose water solution), wherein after the clear aqueous solution is filtered by a 0.22 micron filter, the amount of rolapitant in the filtered aqueous solution is at least 95% of the total amount of rolapitant in the aqueous solution before the filtration. In some embodiments, the composition is a clear aqueous solution when the composition is dissolved in an aqueous solvent (e.g. water, 0.9% saline solution, or 5% dextrose water solution), wherein after the clear aqueous solution is filtered by a 0.22 micron filter, the amount of rolapitant in the filtered aqueous solution is at least 96% of the total amount of rolapitant in the aqueous solution before the filtration. In some embodiments, the composition is a clear aqueous solution when the composition is dissolved in an aqueous solvent (e.g. water, 0.9% saline solution, or 5% dextrose water solution), wherein after the clear aqueous solution is filtered by a 0.22 micron filter, the amount of rolapitant in the filtered aqueous solution is at least 97% of the total amount of rolapitant in the aqueous solution before the filtration. In some embodiments, the composition is a clear aqueous solution when the composition is dissolved in an aqueous solution (e.g. water, 0.9% saline solution, or 5% dextrose water solution), wherein after the clear aqueous solution is filtered by a 0.22 micron filter, the amount of rolapitant in the filtered aqueous solution is at least 98% of the total amount of rolapitant in the aqueous solution before the filtration. In some embodiments, the composition is a clear aqueous solution when the composition is dissolved in an aqueous solution (e.g. water, 0.9% saline solution, or 5% dextrose water solution), wherein after the clear aqueous solution is filtered by a 0.22 micron filter, the amount of rolapitant in the filtered aqueous solution is at least 99% of the total amount of rolapitant in the aqueous solution before the filtration. In some embodiments, the composition is a clear aqueous solution when the composition is dissolved in an aqueous solution (e.g. water, 0.9% saline solution, or 5% dextrose water solution), wherein after the clear aqueous solution is filtered by a 0.22 micron filter, the amount of rolapitant in the filtered aqueous solution is at least 99.5% of the total amount of rolapitant in the aqueous solution before the filtration. In some embodiments, the aqueous solution is free of solvent other than water. In some embodiments, the aqueous solution is substantially free of solvent other than water.

In some embodiments, the amount of rolapitant that is bound to the HSA (e.g., non-covalently) in the aqueous solution (e.g., clear aqueous solution) comprising the composition comprising rolapitant and HSA (as described herein) is at least 40%, at least 50%, at least 60%, at least 70%, at least 80%, at least 90%, at least 95%, at least 99%, or about 100% of the total about of rolapitant in the aqueous solution.

In some embodiments, the composition is a clear aqueous solution when the composition is dissolved in an aqueous solvent (e.g., water, 0.9% saline, or 5% dextrose solution), wherein after the clear aqueous solution is filtered by a 0.22 micron filter, the amount of rolapitant in the filtered aqueous solution is at least 95%, 96%, 97%, 98%, 99%, or 99.5% of the total amount of rolapitant in the aqueous solution before the filtration, wherein the clear aqueous solution has pH value from about 5 to about 8, from about 5.5 to about 7.5, from about 6 to about 7, or from about 6 to about 6.5. In some embodiments, the composition is a clear aqueous solution when the composition is dissolved in an aqueous solvent (e.g., water, 0.9% saline, or 5% dextrose solution), wherein after the clear aqueous solution is filtered by a 0.22 micron filter, the amount of rolapitant in the filtered aqueous solution is at least 95%, 96%, 97%, 98%, 99%, or 99.5% of the total amount of rolapitant in the aqueous solution before the filtration, wherein the clear aqueous solution has pH value from about 6 to about 7.5.

In some embodiments, the composition is a clear aqueous solution when the composition is dissolved in an aqueous solvent (e.g., water, 0.9% saline, or 5% dextrose solution), wherein after the clear aqueous solution is filtered by a 0.22 micron filter after a time period selected from 0 hour, 1 hour, 2 hours, 3 hours, 4 hours, 5 hours, 6 hours and 24 hours, the amount of rolapitant in the filtered aqueous solution is at least 95%, 96%, 97%, 98%, 99%, or 99.5% of the total amount of rolapitant in the aqueous solution at the time of dissolution before the filtration, wherein the clear aqueous solution has pH value from about 5 to about 8, from about 5.5 to about 7.5, from about 6 to about 7, or from about 6 to about 6.5. In some embodiments, the composition is a clear aqueous solution when the composition is dissolved in an aqueous solvent (e.g., water, 0.9% saline, or 5% dextrose solution), wherein after the clear aqueous solution is filtered by a 0.22 micron filter after a time period selected from 0 hour, 1 hour, 2 hours, 3 hours, 4 hours, 5 hours, 6 hours and 24 hours, the amount of rolapitant in the filtered aqueous solution is at least 95%, 96%, 97%, 98%, 99%, or 99.5% of the total amount of rolapitant in the aqueous solution at the time of dissolution before the filtration, wherein the clear aqueous solution has pH value from about 6 to about 7.5.

In some embodiments, the composition is an aqueous solution, wherein after the aqueous solution is filtered by a 0.22 micron filter, the amount of rolapitant in the filtered aqueous solution is at least 80% of the total amount of rolapitant in the aqueous solution before the filtration. In some embodiments, the composition is an aqueous solution, wherein after the aqueous solution is filtered by a 0.22 micron filter, the amount of rolapitant in the filtered aqueous solution is at least 85% of the total amount of rolapitant in the aqueous solution before the filtration. In some embodiments, the composition is an aqueous solution, wherein after the aqueous solution is filtered by a 0.22 micron filter, the amount of rolapitant in the filtered aqueous solution is at least 90% of the total amount of rolapitant in the aqueous solution before the filtration. In some embodiments, the composition is an aqueous solution, wherein after the aqueous solution is filtered by a 0.22 micron filter, the amount of rolapitant in the filtered aqueous solution is at least 95% of the total amount of rolapitant in the aqueous solution before the filtration. In some embodiments, the composition is an aqueous solution, wherein after the aqueous solution is filtered by a 0.22 micron filter, the amount of rolapitant in the filtered aqueous solution is at least 98% of the total amount of rolapitant in the aqueous solution before the filtration. In some embodiments, the composition is an aqueous solution, wherein after the aqueous solution is filtered by a 0.22 micron filter, the amount of rolapitant in the filtered aqueous solution is at least 99% of the total amount of rolapitant in the aqueous solution before the filtration. In some embodiments, the composition is an aqueous solution, wherein after the aqueous solution is filtered by a 0.22 micron filter, the amount of rolapitant in the filtered aqueous solution is 100% of the total amount of rolapitant in the aqueous solution before the filtration. In some embodiments, the aqueous solution is free of solvent other than water. In some embodiments, the aqueous solution is substantially free of solvent other than water.

In some embodiments, the composition is a clear aqueous solution for at least 1 hour, 2 hours, 3 hours, 4 hours, 5 hours, 6 hours, 8 hours, or 24 hours, when the composition is dissolved in an aqueous solvent (e.g., water, 0.9% saline, or 5% dextrose solution). In some embodiments, the aqueous solution is substantially free of solvent other than water. In some embodiments, the aqueous solution free of solvent other than water.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation can be free of a surfactant, such as CREMOPHOR® surfactants and Polysorbate 80. In some embodiments, the aqueous formulation can be substantially free of a surfactant, such as CREMOPHOR® surfactants and Polysorbate 80. In some embodiments, the aqueous formulation can be substantially free of a surfactant selected from the group consisting of CREMOPHOR® surfactants and Polysorbate 80.

As used herein, the term “substantially free of surfactant” refers to a formulation containing less than 0.0005%, less than 0.0003%, or less than 0.0001% of surfactants and/or less than 0.0005%, less than 0.0003%, or less than 0.0001% of surfactant.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

In some embodiments, the aqueous formulation is a clear aqueous solution reconstituted from the solid formulation (e.g. the sterile lyophilized powder) in water. In some embodiments, the aqueous formulation is a clear aqueous solution reconstituted from the solid formulation (e.g. the sterile lyophilized powder) in 0.9% saline. In some embodiments, the aqueous formulation is a clear aqueous solution reconstituted from the solid formulation (e.g. the sterile lyophilized powder) in 5% dextrose solution.

In some embodiments, the aqueous formulation is a clear aqueous solution reconstituted from the solid formulation (e.g. the sterile lyophilized powder) in water, wherein the aqueous formulation has pH value from about 5 to about 8. In some embodiments, the aqueous formulation is a clear aqueous solution reconstituted from the solid formulation (e.g. the sterile lyophilized powder) in water, wherein the pH of water is about 7, and wherein the aqueous formulation has pH value from about 5 to about 8. In some embodiments, the aqueous formulation is a clear aqueous solution reconstituted from the solid formulation (e.g. the sterile lyophilized powder) in 0.9% saline, wherein the aqueous formulation has pH value from about 5 to about 8. In some embodiments, the aqueous formulation is a clear aqueous solution reconstituted from the solid formulation (e.g. the sterile lyophilized powder) in 0.9% saline, wherein the pH of 0.9% saline is about 5.4, and wherein the aqueous formulation has pH value from about 5 to about 8. In some embodiments, the aqueous formulation is a clear aqueous solution reconstituted from the solid formulation (e.g. the sterile lyophilized powder) in 5% dextrose solution, wherein the aqueous formulation has pH value from about 5 to about 8. In some embodiments, the aqueous formulation is a clear aqueous solution reconstituted from the solid formulation (e.g. the sterile lyophilized powder) in 5% dextrose solution, wherein pH of the dextrose solution is about 4.4, and wherein the aqueous formulation has pH value from about 5 to about 8.

In some embodiments, the aqueous formulation is a clear aqueous solution reconstituted from the solid formulation (e.g. the sterile lyophilized powder) in water, wherein the aqueous formulation has pH value from about 6 to about 7.5. In some embodiments, the aqueous formulation is a clear aqueous solution reconstituted from the solid formulation (e.g. the sterile lyophilized powder) in water, wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is a clear aqueous solution reconstituted from the solid formulation (e.g. the sterile lyophilized powder) in 0.9% saline, wherein the aqueous formulation has pH value from about 6 to about 7.5. In some embodiments, the aqueous formulation is a clear aqueous solution reconstituted from the solid formulation (e.g. the sterile lyophilized powder) in 0.9% saline, wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is a clear aqueous solution reconstituted from the solid formulation (e.g. the sterile lyophilized powder) in 5% dextrose solution, wherein the aqueous formulation has pH value from about 6 to about 7.5. In some embodiments, the aqueous formulation is a clear aqueous solution reconstituted from the solid formulation (e.g. the sterile lyophilized powder) in 5% Dextrose solution, wherein the aqueous formulation has pH value of about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some aspects of the aforementioned embodiments, the concentration of the reconstituted solid in the aqueous formulation is from about 25 mg to about 100 mg per 1 mL of the aqueous solvent. In some aspects of the aforementioned embodiments, the concentration of the reconstituted solid in the aqueous formulation is about 25 mg, about 50 mg, about 75 mg, or about 100 mg per 1 ml of the aqueous solvent.

In some embodiments, the aqueous formulation has pH value from about 5 to about 8. In some embodiments, the aqueous formulation has pH value from about 5.5 to about 7.8. In some embodiments, the aqueous formulation has pH value from about 6 to about 7.5. In some embodiments, the aqueous formulation has pH value from about 6.5 to about 7.5. In some embodiments, the aqueous formulation has pH value from about 6 to about 6.5. In some embodiments, the aqueous formulation has pH value from about 6.5 to about 7. In some embodiments, the aqueous formulation has pH value from about 7 to about 7.5. In some embodiments, the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution, wherein the aqueous formulation has pH value from about 5 to about 8, and wherein the aqueous formulation is substantially free of solvent other than water. In some embodiments, the aqueous formulation is a clear aqueous solution, wherein the aqueous formulation has pH value from about 5 to about 8, and wherein the aqueous formulation is free of solvent other than water. In some embodiments, the aqueous formulation is a clear aqueous solution, wherein the aqueous formulation has pH value from about 5.5 to about 7.8, and wherein the aqueous formulation is substantially free of solvent other than water. In some embodiments, the aqueous formulation is a clear aqueous solution, wherein the aqueous formulation has pH value from about 5.5 to about 7.8, and wherein the aqueous formulation is free of solvent other than water. In some embodiments, the aqueous formulation is a clear aqueous solution, wherein the aqueous formulation has pH value from about 6 to about 7.5, and wherein the aqueous formulation is substantially free of solvent other than water. In some embodiments, the aqueous formulation is a clear aqueous solution, wherein the aqueous formulation has pH value from about 6 to about 7.5, and wherein the aqueous formulation is free of solvent other than water. In some embodiments, the aqueous formulation is a clear aqueous solution, wherein the aqueous formulation has pH value from about 6.5 to about 7.5, and wherein the aqueous formulation is substantially free of solvent other than water. In some embodiments, the aqueous formulation is a clear aqueous solution, wherein the aqueous formulation has pH value from about 6.5 to about 7.5, and wherein the aqueous formulation is free of solvent other than water. In some embodiments, the aqueous formulation is a clear aqueous solution, wherein the aqueous formulation has pH value from about 6 to about 6.5, and wherein the aqueous formulation is substantially free of solvent other than water. In some embodiments, the aqueous formulation is a clear aqueous solution, wherein the aqueous formulation has pH value from about 6 to about 6.5, and wherein the aqueous formulation is free of solvent other than water. In some embodiments, the aqueous formulation is a clear aqueous solution, wherein the aqueous formulation has pH value from about 6.5 to about 7, and wherein the aqueous formulation is substantially free of solvent other than water. In some embodiments, the aqueous formulation is a clear aqueous solution, wherein the aqueous formulation has pH value from about 6.5 to about 7, and wherein the aqueous formulation is free of solvent other than water. In some embodiments, the aqueous formulation is a clear aqueous solution, wherein the aqueous formulation has pH value from about 7 to about 7.5, and wherein the aqueous formulation is substantially free of solvent other than water. In some embodiments, the aqueous formulation is a clear aqueous solution, wherein the aqueous formulation has pH value from about 7 to about 7.5, and wherein the aqueous formulation is free of solvent other than water. In some embodiments, the aqueous formulation is a clear aqueous solution, wherein the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5, and wherein the aqueous formulation is substantially free of solvent other than water. In some embodiments, the aqueous formulation is a clear aqueous solution, wherein the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5, and wherein the aqueous formulation is free of solvent other than water.

In some embodiments, the aqueous formulation is a clear aqueous solution for at least 1 hour. In some embodiments, the aqueous formulation is a clear aqueous solution for at least 1 hour at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 2 hours. In some embodiments, the aqueous formulation is a clear aqueous solution for at least 2 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 hours. In some embodiments, the aqueous formulation is a clear aqueous solution for at least 3 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 6 hours. In some embodiments, the aqueous formulation is a clear aqueous solution for at least 6 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 24 hours. In some embodiments, the aqueous formulation is a clear aqueous solution for at least 24 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 days. In some embodiments, the aqueous formulation is a clear aqueous solution for at least 3 days when dissolved in an aqueous solution at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least about 1 hour, 2 hours, 3 hours, 4 hours, 5 hours, 6 hours, 8 hours, or 24 hours. In some embodiments, the aqueous formulation is substantially free of solvent other than water. In some embodiments, the aqueous formulation is free of solvent other than water.

In some embodiments, after the aqueous formulation is filtered by a 0.22 micron filter, the amount of rolapitant in the filtered aqueous solution is at least 95% of the total amount of rolapitant in the aqueous solution before the filtration. In some embodiments, after the aqueous formulation is filtered by a 0.22 micron filter, the amount of rolapitant in the filtered aqueous solution is at least 96% of the total amount of rolapitant in the aqueous solution before the filtration. In some embodiments, after the aqueous formulation is filtered by a 0.22 micron filter, the amount of rolapitant in the filtered aqueous solution is at least 97% of the total amount of rolapitant in the aqueous solution before the filtration. In some embodiments, after the aqueous formulation is filtered by a 0.22 micron filter, the amount of rolapitant in the filtered aqueous solution is at least 98% of the total amount of rolapitant in the aqueous solution before the filtration. In some embodiments, after the aqueous formulation is filtered by a 0.22 micron filter, the amount of rolapitant in the filtered aqueous solution is at least 99% of the total amount of rolapitant in the aqueous solution before the filtration. In some embodiments, after the aqueous formulation is filtered by a 0.22 micron filter, the amount of rolapitant in the filtered aqueous solution is at least 99.5% of the total amount of rolapitant in the aqueous solution before the filtration. In some aspects of these embodiments, the aqueous formulation is filtered by a 0.22-micron filter at a time period selected from 0 hour, 1 hour, 2 hours, 3, hours, 4 hours, 5 hours, 6 hours, 8 hours, 12 hours, 18 hours and 24 hours. In some embodiments, the aqueous formulation is free of solvent other than water. In some embodiments, the aqueous formulation is substantially free of solvent other than water.

In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 5 to about 8, and wherein the aqueous formulation is substantially free of solvent other than water.

In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 6 to about 7.5, and wherein the aqueous formulation is substantially free of solvent other than water. In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 5 to about 8, and wherein the aqueous formulation is free of solvent other than water. In some embodiments, after the aqueous formulation (e.g. a clear aqueous solution) is filtered by a 0.22 micron filter, the amount of rolapitant in the filtered aqueous solution is at least 95%, 96%, 97%, 98%, 99%, or 99.5% of the total amount of rolapitant in the aqueous solution before filtration, wherein the aqueous formulation has pH value from about 6 to about 7.5, and wherein the aqueous formulation is free of solvent other than water. In some aspects of these embodiments, the aqueous formulation is filtered by a 0.22-micron filter at a time period selected from 0 hour, 1 hour, 2 hours, 3, hours, 4 hours, 5 hours, 6 hours, 8 hours, 12 hours, 18 hours and 24 hours.

Also, provided herein is a pharmaceutical composition comprising the composition comprising the rolapitant and the human serum albumin as described herein, and a pharmaceutically acceptable carrier.

In some embodiments, the pharmaceutical composition comprising the composition comprising the rolapitant and the human serum albumin as described herein, and a pharmaceutically acceptable carrier, is a liquid pharmaceutical composition. In some embodiments, the liquid pharmaceutical composition is an aqueous solution. In some embodiments, the liquid pharmaceutical composition is free of solvent other than water. In some embodiments, the liquid pharmaceutical composition is substantially free of solvent other than water. In some embodiments, the liquid pharmaceutical composition is an injectable pharmaceutical formulation. In some embodiments, the liquid pharmaceutical composition is a formulation for infusion (e.g., intravenous infusion).

In some embodiments, the pharmaceutically acceptable carrier is refers to any carrier useful to solubilize and deliver an agent to a subject. A desirable pharmaceutically acceptable carrier is saline. Other pharmaceutically acceptable carrier and their formulation are known to one skilled in the art and described, for example, in Remington's Pharmaceutical Sciences. (20^{th} edition), ed. A. Gennaro, 2003, Lippincon Williams & Wilkins. In some embodiments, the carrier may contain components such as, for example, dextrose, glucose, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, and cellulose-based substances. In some embodiments, the carrier may contain components such as contain anti-oxidants, buffers, bacteriostats and solutes which render the formulation isotonic with the blood of the intended recipient.

In some embodiments, the pharmaceutically acceptable excipient is selected from lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical compositions may additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The pharmaceutical composition may be formulated so as to provide quick, sustained or delayed release of the active ingredient after administration to the patient by employing procedures known in the art.

In some embodiments, the pharmaceutical composition of the present disclosure may be administered by a syringe or a catheter, or any other means generally known in the art for the delivery of a pharmaceutical agent by injection to the subject in need thereof. The delivery means will vary, as recognized by those skilled in the art, depending on the diseases and conditions treated, the severity of the disease, the sex, age and general health condition of the subject, excipient usage, the possibility of co-usage with other therapeutic treatments such as use of other agents as described herein and the judgment of the treating physician.

The pharmaceutical composition comprising the composition comprising the rolapitant and the human serum albumin as described herein can be administered to a subject via various routes, such as parenterally, including intravenous, intra-arterial, intraperitoneal, intrapulmonary, oral, inhalation, intravascular, intramuscular, intra-tracheal, subcutaneous, intraocular, intrathecal, or transdermal. For example, the composition can be administered by inhalation to treat conditions of the respiratory tract. In some embodiments, the pharmaceutical composition is administrated in a unit dosage form such as capsule or oral suspension (e.g., for pediatric patients 6 months to less than 12 years of age).

In some embodiments, the pharmaceutical composition is free of a vehicle selected from the group consisting of water soluble organic solvents, non-ionic surfactants, water insoluble lipids, organic lipids/semisolids and phospholipids. Water soluble organic solvents may be selected from, for example, polyethylene glycol 300, polyethylene glycol 400, ethanol, propylene glycol, glycerin, N-methyl-2-pyrrolidone, dimethylacetamide and dimethylsulfoxide. Non-ionic surfactants may be selected from Cremophor EL, Cremophor RH 40, Cremophor RH 60, d-a-tocopherol polyethylene glycol 1000 succinate, polysorbate 80, Solutol HS 15, sorbitan monooleate, poloxamer 407, Labrifil M-1944CS, Labrafil M-2125CS, Labrasol, Gellucire 44/14, Softigen 767, and mono- and di-fatty acid esters of PEG 300, 400 or 1750. The water insoluble lipids are selected from castor oil, corn oil, cottonseed oil, olive oil, peanut oil, peppermint oil, safflower oil, sesame oil, soybean oil, hydrogenated vegetable oils, hydrogenated soybean oil, and medium chain triglycerides of coconut oil and palm seed oil. Organic liquids and semisolids may be selected from beeswax, d-a-tocopherol, oleic acid and medium chain mono- and diglycerides. The phospholipids may be selected from lecithin, hydrogenated soy phosphatidylcholine, distearoylphosphatidylglycerol, L-a-dimyristoylphosphatidylcholine and L-a-dimyristoylphosphatidylglycerol.

In some embodiments, the pharmaceutical composition is free of macrogol 15-hydroxystearate. In some embodiments, the pharmaceutical composition is free of a medium chain triglyceride. In some embodiments, the pharmaceutical composition is free of a long chain triglyceride. In some embodiments, the pharmaceutical composition is free of soybean oil. In some embodiments, the pharmaceutical composition can be substantially free of a vehicle selected from the group consisting of macrogol 15-hydroxystearate, a medium chain triglyceride, a long chain triglyceride, and soybean oil. In some embodiments, the pharmaceutical composition can be free of a vehicle selected from the group consisting of macrogol 15-hydroxystearate, a medium chain triglyceride, a long chain triglyceride, and soybean oil.

In some embodiments, the pharmaceutical composition is free of a surfactant, such as CREMOPHOR® surfactants and Polysorbate 80. In some embodiments, the pharmaceutical composition is substantially free of a surfactant, such as CREMOPHOR® surfactants and Polysorbate 80. In some embodiments, the pharmaceutical composition can be substantially free of a surfactant selected from the group consisting of CREMOPHOR® surfactants and Polysorbate 80.

Also, provided herein is a method for the prevention of chemotherapy-induced nausea and vomiting (CINV) or postoperative nausea and vomiting (PONV), the method comprising the step of administering to a subject in need thereof of a therapeutically effective amount of a pharmaceutical composition comprising the composition comprising the rolapitant and the human serum albumin as described herein, and a pharmaceutically acceptable carrier. In some embodiments, the methods described herein are for the prevention of chemotherapy-induced nausea and vomiting (CINV). In some embodiments, the methods described herein are for the prevention of delayed nausea and vomiting associated with initial and repeat courses of emetogenic cancer chemotherapy. Including, but not limited to, highly emetogenic cancer chemotherapy (HEC). In some embodiments, the methods described herein are for the prevention of nausea and vomiting associated with initial and repeat courses of highly emetogenic cancer chemotherapy (HEC). In some embodiments, the methods described herein are for the prevention of nausea and vomiting associated with initial and repeat courses of moderately emetogenic cancer chemotherapy (MEC). In some embodiments, the methods described herein are for the prevention of postoperative nausea and vomiting (PONV). In some embodiments, the methods described herein are for the prevention of cough (e.g., chronic cough from unknown cause).

As used herein, the term “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, prevention of a disease, condition or disorder refers to decreasing the possibility of acquiring the disease, condition or disorder and/or its associated symptoms. In some embodiments, prevention of a disease, condition or disorder refers to completely or almost completely stopping the disease, condition or disorder from occurring.

As used herein, the terms “individual”, “patient”, or “subject” are used interchangeably and refer to any animal, including mammals, preferably mice, rats, other rodents, rabbits, dogs, cats, swine, cattle, sheep, horses, or primates, and most preferably humans.

As used herein, an “effective amount,” “therapeutically effective amount,” or a “pharmaceutically-effective amount” in reference to the compounds or compositions of the instant invention refers to the amount sufficient to induce a desired biological, pharmacological, or therapeutic outcome in a subject. That result can be reduction, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In cancer treatment, the result will generally include the reduction, mitigation, limitation, and/or, delay of the deleterious physiological manifestations, growth or metastases of neoplasms.

Also, provided herein is a method of treating chemotherapy-induced nausea and vomiting, the method comprising the step of administering to a subject in need thereof of a therapeutically effective amount of a pharmaceutical composition comprising the composition comprising the rolapitant and the human serum albumin as described herein, and a pharmaceutically acceptable carrier. In some aspects of these embodiments, the chemotherapy-induced nausea and vomiting is delayed nausea and vomiting associated with initial and repeat courses of emetogenic cancer chemotherapy. Including, but not limited to, highly emetogenic cancer chemotherapy (HEC). In other aspects of these embodiments, the chemotherapy-induced nausea and vomiting is nausea and vomiting associated with initial and repeat courses of moderately emetogenic cancer chemotherapy (MEC). In other aspects of these embodiments, the chemotherapy-induced nausea and vomiting is nausea and vomiting associated with initial and repeat courses of highly emetogenic cancer chemotherapy (HEC). In some embodiments, the methods described herein are for the treating cough (e.g., chronic cough from unknown cause).

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

In some embodiments, the methods described herein are performed in combination with at least one other antiemetic agents. In some embodiments, the methods described herein are performed in combination with dexamethasone. In some embodiments, the methods described herein are performed in combination with dexamethasone and a 5-HT3 antagonist (e.g., tropisetron, palonosetron, ramosetron, granisetron, ondansetron, dolasetron, or metoclopramide). In some embodiments, the antiemetic agent is selected from tropisetron, palonosetron, ramosetron, granisetron, ondansetron, dolasetron, metoclopramide, domperidone, olanzapine, droperidol, haloperidol, chlorpromazine, prochlorperazine, alizapride, prochlorperazine, metoclopramide, casopitant, cyclizine, diphenhydramine, dimenhydrinate, doxylamine, meclizine, promethazine, hydroxyzine, dronabinol, sativex, midazolam, lorazepam, hyoscine, trimethobenzamide, emetrol, propofol and muscimol.

In some embodiments, provided herein is a method for preventing chemotherapy-induced nausea and vomiting (e.g., acute and delayed nausea and vomiting associated with initial and repeat courses of emetogenic cancer chemotherapy. Including, but not limited to, highly emetogenic cancer chemotherapy (HEC); or of delayed nausea and vomiting associated with initial and repeat courses of highly emetogenic cancer chemotherapy (HEC), or of delayed nausea and vomiting associated with initial and repeat courses of highly emetogenic cancer chemotherapy (HEC), or postoperative nausea and vomiting (PONV) in adults), the method comprising administering to a subject in need thereof a therapeutically effective amount a pharmaceutical composition comprising the composition comprising the rolapitant and the human serum albumin as described herein, and a therapeutically effective amount of at least one antiemetic agent as described herein.

As used herein, the term “emetogenic chemotherapy” refers to chemotherapy during which a patient experiences emesis in the absence of effective antiemetic prophylaxis. Level 1 emetogenic chemotherapy refers to chemotherapy in which less than 10% of patients experience emesis; level 2 emetogenic chemotherapy refers to chemotherapy in which 10-30% of patients experience emesis; level 3 emetogenic chemotherapy refers to chemotherapy in which 30-60% of patients experience emesis; level 4 emetogenic chemotherapy refers to chemotherapy in which 60-90% of patients experience emesis; and level 5 emetogenic chemotherapy refers to chemotherapy in which more than 90% of patients experience emesis in the absence of prophylaxis. In some embodiments, level 3-5 cancer chemotherapy highly emetogenic (HEC). In some embodiments, level 1-2 cancer chemotherapy moderately emetogenic (MEC).

In some embodiments, emetogenic chemotherapy is cisplatin-based or taxane-based chemotherapy. In some embodiments, emetogenic chemotherapy is a therapy with an anti-cancer agent selected from carmustine, cisplatin, cyclophosphamide, dacarbazine, mechlorethamine, streptozocin, carboplatin, cytarabine, doxorubicin, methotrexate, procarbazine, cyclophosphamid, epirubicin, hexamethylmelamine, idarubicin, ifosfamide, irinotecan, mitoxantrone, capecitabine, docetaxel, etoposide, 5-fluorouracil, gemcitabine, mitomycin, paclitaxel, topotecan, bleomycin, busulfan, chlorambucil, 2-chlorodeoxyadenosine, fludarabine, hydroxyurea, L-phenylalanine mustard, thioguanine, vinblastine, vincristine and vinorelbine.

In some embodiments, a pharmaceutical composition comprising the composition comprising the rolapitant and the human serum albumin as described herein and an antiemetic agent are administered simultaneously.

In some embodiments, a pharmaceutical composition comprising the composition comprising the rolapitant and the human serum albumin as described herein and an antiemetic agent are administered consecutively.

Also, provided herein is a pharmaceutical composition comprising the composition comprising the rolapitant and the human serum albumin as described herein, at least one antiemetic agent as described herein, and a pharmaceutically acceptable carrier.

In some embodiments, provided herein is a pharmaceutical composition comprising the composition comprising the rolapitant and the human serum albumin as described herein, a 5-HT3 antagonist (e.g., tropisetron, palonosetron, ramosetron, granisetron, ondansetron, dolasetron, or metoclopramide), and a pharmaceutically acceptable carrier.

In some embodiments, provided herein is a pharmaceutical composition comprising the composition comprising the rolapitant and the human serum albumin as described herein, palonosetron, and a pharmaceutically acceptable carrier.

In some embodiments, provided herein is a pharmaceutical composition comprising the composition comprising the rolapitant and the human serum albumin as described herein, 0.5 mg of palonosetron, and a pharmaceutically acceptable carrier.

In some embodiments, provided herein is a pharmaceutical composition comprising the composition comprising the rolapitant and the human serum albumin as described herein, 0.25 mg of palonosetron, and a pharmaceutically acceptable carrier.

In some embodiments, provided herein is a pharmaceutical composition comprising the composition comprising the rolapitant and the human serum albumin as described herein, 0.28 mg of palonosetron hydrochloride, and a pharmaceutically acceptable carrier.

In some embodiments, provided herein is a pharmaceutical composition comprising the composition comprising the rolapitant and the human serum albumin as described herein, about 0.25 mg of palonosetron, and a pharmaceutically acceptable carrier.

In some embodiments, provided herein is a pharmaceutical composition comprising the composition comprising the rolapitant and the human serum albumin as described herein, about 0.28 mg of palonosetron hydrochloride, and a pharmaceutically acceptable carrier.

In some embodiments, provided herein is a liquid pharmaceutical formulation for injection comprising the composition comprising the rolapitant and the human serum albumin as described herein, 0.25 mg of palonosetron, and a pharmaceutically acceptable carrier.

In some embodiments, provided herein is a liquid pharmaceutical formulation for injection comprising the composition comprising the rolapitant and the human serum albumin as described herein, 0.28 mg of palonosetron hydrochloride, and a pharmaceutically acceptable carrier.

In some embodiments, the liquid pharmaceutical formulation for injection is an aqueous solution. In some embodiments, the liquid pharmaceutical formulation for injection is free of solvent other than water. In some embodiments, the liquid pharmaceutical formulation for injection is substantially free of solvent other than water. In some embodiments, the liquid pharmaceutical formulation for injection is a formulation for infusion (e.g., intravenous infusion).

In some embodiments, the liquid pharmaceutical formulation for injection has pH value from about 5 to about 8. In some embodiments, the liquid pharmaceutical formulation for injection has pH value from about 5.5 to about 7.8. In some embodiments, the liquid pharmaceutical formulation for injection has pH value from about 6 to about 7.5. In some embodiments, the liquid pharmaceutical formulation for injection has pH value from about 6.5 to about 7.5. In some embodiments, the liquid pharmaceutical formulation for injection has pH value from about 6 to about 6.5. In some embodiments, the liquid pharmaceutical formulation for injection has pH value from about 6.5 to about 7. In some embodiments, the liquid pharmaceutical formulation for injection has pH value from about 7 to about 7.5. In some embodiments, the liquid pharmaceutical formulation for injection has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the liquid pharmaceutical formulation for injection is substantially free of solvent other than water. In some embodiments, the liquid pharmaceutical formulation for injection is free of solvent other than water.

As used herein the term “palonosetron” is a compound that has the CAS No. 135729-61-2 and the following chemical structure:

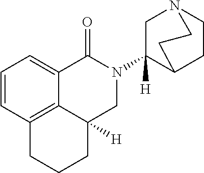 [SMILES: O=C1c2cccc3c2[C@H](CCC3)CN1[C@@H]1CN2CCC1CC2]

In some embodiments, the palonosetron can be a pharmaceutically acceptable salt of palonosetron. In some embodiments, the palonosetron can be palonosetron hydrochloride.

As used herein the term “palonosetron hydrochloride” is a compound that has the CAS No. 135729-62-3 and the following chemical structure:

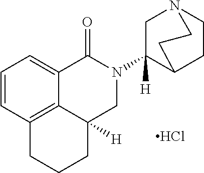 [SMILES: O=C1c2cccc3c2[C@H](CCC3)CN1[C@@H]1CN2CCC1CC2 ; Cl]

In some embodiments, provided herein is a pharmaceutical composition comprising the composition comprising the rolapitant and the human serum albumin as described herein, granisetron, and a pharmaceutically acceptable carrier.

In some embodiments, provided herein is a pharmaceutical composition comprising the composition comprising the rolapitant and the human serum albumin as described herein, ondansetron, and a pharmaceutically acceptable carrier.

The methods described herein may be performed alone or in conjunction with another therapy, such as surgery, radiation, chemotherapy, immunotherapy, gene therapy, and the like. In some embodiments, a composition comprising the rolapitant and the human serum albumin as described herein is administered prior to chemotherapy.

In some embodiments, the amount of rolapitant that is administered to a subject in need thereof with any one of pharmaceutical compositions described herein is from about 50 mg to about 250 mg, from about 60 mg to about 240 mg, from about 70 mg to 230 mg, from about 80 mg to about 220 mg, from about 90 mg to about 210 mg, from about 100 mg to about 200 mg, or from about 100 mg to about 180 mg. In some embodiments, the amount of rolapitant that is administered to a subject in need thereof with any one of pharmaceutical compositions described herein is about 100 mg, about 125 mg, about 150 mg, about 160, about 165 mg, about 170, about 175, about 180 mg, about 185 mg, about 190 mg, about 200 mg, about 210 mg, about 220 mg, about 225 mg, or about 250 mg.

In some embodiments, a composition comprising the rolapitant and the human serum albumin as described herein is administered IV (intravenously). In some embodiments, a composition comprising the rolapitant and the human serum albumin as described herein is administered as an infusion in a dosage from about 0.1 mg/ml to about 20 mg/ml, from about 0.2 mg/ml to about 15 mg/ml, from about 0.5 mg/ml to about 12 mg/ml, from about 0.6 mg/ml to about 10 mg/ml, from about 0.7 mg/ml to about 8 mg/ml, from about 0.8 mg/ml to about 6 mg/ml, from about 0.9 mg/ml to about 5 mg/ml, or from about 1 mg/ml to about 3 mg/ml. In some embodiments, a composition comprising the rolapitant and the human serum albumin as described herein is administered as an infusion in a dosage of about 0.2 mg/ml, 0.5 mg/ml, about 0.75 mg/ml, about 1 mg/ml, about 1.25 mg/ml, about 1.5 mg/ml, about 1.75 mg/ml, about 2 mg/ml, about 2.25 mg/ml, about 2.5 mg/ml, about 2.75 mg/ml, about 3 mg/ml, about 4 mg/ml, or about mg/ml. In some embodiments, a composition comprising the rolapitant and the human serum albumin as described herein is administered as an infusion for a time period from about 1 min to about 24 hours, from about 5 min to about 18 hours, from about 10 min to about 14 hours, from about 15 min to about 12 hours, from about 20 min to about 10 hours, from about 30 min to about 10 hours, or from about 1 hour to about 8 hours. In some embodiments, a composition comprising the rolapitant and the human serum albumin as described herein is administered as an infusion for a time period of about 10 min, about 15 min, about 30 min, about 45 min, about 1 hour, about 3 hours, about 5 hours, or about 10 hours.

As will be understood by those of ordinary skill in the art, the appropriate doses of rolapitant will be approximately those already employed in clinical therapies wherein rolapitant is administered alone or in combination with other therapeutic agents. Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician. For example, guidance for selecting an effective dose can be determined by reference to the prescribing information for rolapitant.

Also, provided herein is a composition consisting essentially of rolapitant and human serum albumin, wherein the rolapitant and the human serum albumin in the composition have a ratio by weight from about 1:20 to about 1:2000.

In some embodiments, the rolapitant and the human serum albumin in the composition have a ratio by weight from about 1:100 to about 1:1000, from about 1:130 to about 1:800, from about 1:140 to about 1:600, about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:175, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

In some embodiments, the rolapitant can be a pharmaceutically acceptable salt of rolapitant. In some embodiments, rolapitant is rolapitant hydrochloride. In some embodiments, rolapitant can be any one of crystal forms, amorphous forms, solvates and hydrates as described herein. In some embodiments, rolapitant hydrochloride can be any one of crystal forms, amorphous forms, solvates and hydrates as described herein.

In some embodiments, the composition is a clear aqueous solution when the composition is dissolved in an aqueous solution. In some embodiments, the aqueous solution is substantially free of solvent other than water. In some embodiments, the aqueous solution is free of solvent other than water.

As used herein, the term “clear aqueous solution” refers to an aqueous solution containing rolapitant and HSA that is transparent upon visual observation and essentially free of visible particles or precipitation of undissolved rolapitant.

The term “essentially free of visible particles or precipitation of undissolved rolapitant” can be assessed as follows: after a clear aqueous solution is filtered with a 0.22 micron filter, the amount of rolapitant in the filtered aqueous solution is at least 95% of the total amount of rolapitant in the aqueous solution before filtration. The total amount of rolapitant in the aqueous solution before filtration includes the particles or precipitation of undissolved rolapitant in the aqueous solution or with the aqueous solution. The amount of the rolapitant in an aqueous solution can be measured by the methods using HPLC. The methods of measuring the amount of the rolapitant in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

In some embodiments, the composition is a solid formulation. For example, the solid formulation can be produced in a uniform manner by lyophilization. A skilled artisan would recognize other methods, such as rotary evaporation, that can also produce solid formulations.

In some embodiments, the composition is an aqueous formulation. In some embodiments, the aqueous formulation is substantially free of solvent other than water. In some embodiments, the aqueous formulation is free of solvent other than water.

In some embodiments, the aqueous formulation can be free of a surfactant, such as CREMOPHOR® surfactants and Polysorbate 80. In some embodiments, the aqueous formulation can be substantially free of a surfactant, such as CREMOPHOR® surfactants and Polysorbate 80. In some embodiments, the aqueous formulation can be substantially free of a surfactant selected from the group consisting of CREMOPHOR® surfactants and Polysorbate 80.

As used herein, the term “substantially free of surfactant” refers to a formulation containing less than 0.0005%, less than 0.0003%, or less than 0.0001% of surfactants and/or less than 0.0005%, less than 0.0003%, or less than 0.0001% of surfactant.

In some embodiments, the aqueous formulation is a clear aqueous solution. For example, the formulation can be a clear and stable aqueous solution reconstituted from a sterile lyophilized powder. In some embodiments, the aqueous formulation is a clear aqueous solution, wherein the aqueous formulation is substantially free of solvent other than water. In some embodiments, the aqueous formulation is a clear aqueous solution, wherein the aqueous formulation is free of solvent other than water.

In some embodiments, the aqueous formulation has pH value from about 5 to about 8. In some embodiments, the aqueous formulation has pH value from about 5.5 to about 7.8. In some embodiments, the aqueous formulation has pH value from about 6 to about 7.5. In some embodiments, the aqueous formulation has pH value from about 6.5 to about 7.5. In some embodiments, the aqueous formulation has pH value from about 6 to about 6.5. In some embodiments, the aqueous formulation has pH value from about 6.5 to about 7. In some embodiments, the aqueous formulation has pH value from about 7 to about 7.5. In some embodiments, the aqueous formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous formulation is substantially free of solvent other than water. In some embodiments, the aqueous formulation is free of solvent other than water.

In some embodiments, the aqueous formulation is a clear aqueous solution for at least 1 hour. In some embodiments, the aqueous formulation is a clear aqueous solution for at least 1 hour at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 2 hours. In some embodiments, the aqueous formulation is a clear aqueous solution for at least 2 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 hours. In some embodiments, the aqueous formulation is a clear aqueous solution for at least 3 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 6 hours. In some embodiments, the aqueous formulation is a clear aqueous solution for at least 6 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 24 hours. In some embodiments, the aqueous formulation is a clear aqueous solution for at least 24 hours at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least 3 days. In some embodiments, the aqueous formulation is a clear aqueous solution for at least 3 days when dissolved in an aqueous solution at a temperature from about 1° C. to about 35° C., about 1° C. to about 10° C., about 10° C. to about 20° C., about 20° C. to about 35° C., or about 1° C., about 5° C., about 10° C., about 15° C., about 20° C., about 25° C., about 30° C., or about 35° C. In some embodiments, the aqueous formulation is a clear aqueous solution for at least about 1 hour, 2 hours, 3 hours, 4 hours, 5 hours, 6 hours, 8 hours, or 24 hours. In some embodiments, the aqueous formulation is substantially free of solvent other than water. In some embodiments, the aqueous formulation is free of solvent other than water.

Also, provided herein is a pharmaceutical composition comprising the composition consisting essentially of the rolapitant and the human serum albumin as described herein, and a pharmaceutically acceptable carrier.

Also, provided herein is a method for the prevention of chemotherapy-induced nausea and vomiting or postoperative nausea and vomiting (PONV), the method comprising the step of administering to a subject in need thereof of a therapeutically effective amount of a pharmaceutical composition comprising the composition comprising the rolapitant and the human serum albumin as described herein, and a pharmaceutically acceptable carrier. In some embodiments, the methods described herein are for the prevention of chemotherapy-induced nausea and vomiting. In some embodiments, the methods described herein are for the prevention of delayed nausea and vomiting associated with initial and repeat courses of emetogenic cancer chemotherapy. Including, but not limited to, highly emetogenic cancer chemotherapy (HEC). In some embodiments, the methods described herein are for the prevention of nausea and vomiting associated with initial and repeat courses of highly emetogenic cancer chemotherapy (HEC). In some embodiments, the methods described herein are for the prevention of nausea and vomiting associated with initial and repeat courses of moderately emetogenic cancer chemotherapy (MEC). In some embodiments, the methods described herein are for the prevention of postoperative nausea and vomiting (PONV).

Also, provided herein is a method of treating chemotherapy-induced nausea and vomiting, the method comprising the step of administering to a subject in need thereof of a therapeutically effective amount of a pharmaceutical composition comprising the composition consisting essentially of the rolapitant and the human serum albumin as described herein, and a pharmaceutically acceptable carrier. In some aspects of these embodiments, the chemotherapy-induced nausea and vomiting is delayed nausea and vomiting associated with initial and repeat courses of emetogenic cancer chemotherapy. Including, but not limited to, highly emetogenic cancer chemotherapy (HEC). In other aspects of these embodiments, the chemotherapy-induced nausea and vomiting is nausea and vomiting associated with initial and repeat courses of moderately emetogenic cancer chemotherapy (MEC). In other aspects of these embodiments, the chemotherapy-induced nausea and vomiting is nausea and vomiting associated with initial and repeat courses of highly emetogenic cancer chemotherapy (HEC).

In some embodiments, the methods described herein are performed in combination with at least one other antiemetic agents. In some embodiments, the methods described herein are performed in combination with dexamethasone and a 5-HT3 antagonist (e.g., tropisetron, palonosetron, ramosetron, granisetron, ondansetron, dolasetron, or metoclopramide). In some embodiments, the antiemetic agent is selected from tropisetron, palonosetron, ramosetron, granisetron, ondansetron, dolasetron, metoclopramide, domperidone, olanzapine, droperidol, haloperidol, chlorpromazine, prochlorperazine, alizapride, prochlorperazine, metoclopramide, casopitant, cyclizine, diphenhydramine, dimenhydrinate, doxylamine, meclizine, promethazine, hydroxyzine, dronabinol, sativex, midazolam, lorazepam, hyoscine, trimethobenzamide, emetrol, propofol and muscimol.

In some embodiments, provided herein is a method for preventing chemotherapy-induced nausea and vomiting (e.g., acute and delayed nausea and vomiting associated with initial and repeat courses of emetogenic cancer chemotherapy. Including, but not limited to, highly emetogenic cancer chemotherapy (HEC); or of delayed nausea and vomiting associated with initial and repeat courses of highly emetogenic cancer chemotherapy (HEC), or of delayed nausea and vomiting associated with initial and repeat courses of highly emetogenic cancer chemotherapy (HEC), or postoperative nausea and vomiting (PONV) in adults), the method comprising administering to a subject in need thereof a therapeutically effective amount a pharmaceutical composition comprising the composition consisting essentially of the rolapitant and the human serum albumin as described herein, and a therapeutically effective amount of at least one antiemetic agent as described herein.

Kits

The present invention also includes pharmaceutical kits useful, for example, in the treatment or prevention of any one of diseases or disorders referred to herein, which include one or more containers containing a pharmaceutical composition comprising a composition of rolapitant and the human serum albumin as described herein. Such kits can further include, if desired, one or more of various conventional pharmaceutical kit components, such as, for example, containers with one or more pharmaceutically acceptable carriers (e.g., water, saline (e.g., 0.9% saline), or 5% dextrose), additional containers, etc., as will be readily apparent to those skilled in the art. Instructions, either as inserts or as labels, indicating quantities of the components to be administered (e.g., dosage amounts as described herein), guidelines for administration, and/or guidelines for mixing the components, can also be included in the kit.

### Methods of Making

Also, provided herein are several methods to prepare a composition comprising a non-covalently bound complex comprising the rolapitant and the human serum albumin as described herein, a composition comprising the rolapitant and the human serum albumin as described herein, or a composition consisting essentially of the rolapitant and the human serum albumin as described herein.

In some embodiments, the present disclosure provides a method of preparing a composition comprising a non-covalently bound complex comprising rolapitant and human serum albumin, wherein the rolapitant and the human serum albumin in the composition have a ratio by weight from about 1:20 to about 1:2000.

In some embodiments, the present disclosure provides a method of preparing a composition comprising a non-covalently bound complex comprising rolapitant and human serum albumin, wherein the rolapitant and the human serum albumin in the complex have a ratio by weight from about 1:20 to about 1:2000.

In some embodiments, the present disclosure provides a method of preparing a composition comprising rolapitant and human serum albumin, wherein the rolapitant and the human serum albumin in the composition have a ratio by weight from about 1:20 to about 1:2000.

In some embodiments, the present disclosure provides a method of preparing a composition consisting essentially of rolapitant and human serum albumin, wherein the rolapitant and the human serum albumin in the composition have a ratio by weight from about 1:20 to about 1:2000.

In some embodiments, the method comprises mixing an organic solution of rolapitant in a polar water-miscible organic solvent and a first aqueous solution containing human serum albumin to form a second aqueous solution, wherein the second aqueous solution is a clear aqueous solution.

In some embodiments, the method further comprises removing said polar water-miscible organic solvent and water from the second aqueous solution.

In some embodiments, the method comprises the steps of:

(i) obtaining an organic solution of rolapitant in a polar water-miscible organic solvent;

(ii) obtaining a first aqueous solution of human serum albumin; and

(iii) mixing the organic solution of rolapitant and the first aqueous solution of human serum albumin to obtain a second aqueous solution comprising the composition comprising rolapitant and human serum albumin as described herein.

A non-limiting embodiments of the method are as follows.

### Formation of the Organic Solution

In some embodiments, rolapitant is dissolved in a polar organic solvent (e.g., an alcohol such as methanol, ethanol, isopropanol, and/or n-butanol; THF, CH₃CN; DMF; or mixtures thereof) to form an organic solution.

As used herein, the term “organic solution” refers to a solution wherein at least one solvent is a non-aqueous solvent and the weight % of the non-aqueous solvent in the mixture of solvents is at least 50%, at least 60%, at least 70% or at least 90%. In some embodiments, organic solution is a solution in which does not comprise water as a solvent.

In some embodiments, the terms “organic solvent” and “non-aqueous solvent” are used interchangeably and refer to a liquid comprising is at least 50%, at least 60%, at least 70%, at least 90%, or at least 95% of a solvent other than water. In some embodiments, organic solvent is polar (e.g., polar aprotic solvent such as tetrahydrofuran, ethyl acetate, acetone, dimethylformamide, acetonitrile, dimethyl sulfoxide or nitromethane; or a polar protic solvent such as an alcohol, or an acid such as formic acid or an acetic acid). In some embodiments, the organic solvent is water-miscible (i.e., can be mixed with water in all proportions) or water-immiscible (i.e., significant proportions of organic solvent/water do not form a solution).

In some embodiments, the organic solvent is polar organic solvent that is miscible in water (e.g., tetrahydrofuran, propylene glycol, propanol, methanol, ethanol, dimethyl sulfoxide, dimethylformamide, acetonitrile or acetone). In some embodiments, the polar organic solvent is an alcohol. In some embodiments, the polar organic solvent is ethanol or methanol, or mixtures thereof. In some embodiments, the polar organic solvent can be ethanol. In some embodiments, the polar organic solvent is methanol.

In some embodiments, the amount of polar organic solvent (e.g., methanol) is from about 0.005 mL to about 10 mL per 1 mg of rolapitant. In some embodiments, the amount of polar organic solvent is from about 0.01 mL to about 5 mL per 1 mg of rolapitant. In some embodiments, the amount of polar organic solvent is from about 0.05 mL to about 5 mL per 1 mg of rolapitant. In some embodiments, the amount of polar organic solvent is from about 0.1 mL to about 3.0 mL per 1 mg of rolapitant. In some embodiments, the amount of polar organic solvent is from about 0.2 mL to about 2.0 mL per 1 mg of rolapitant. In some embodiments, the amount of polar organic solvent is from about 0.5 mL to about 4.0 mL, from about 0.6 mL to about 3.9 mL, from about 0.7 mL to about 3.8 mL, from about 0.8 mL to about 3.7 mL, from about 0.9 mL to about 3.6 mL, from about 1.0 mL to about 3.0 mL, or from about 1.1 mL to about 2.6 mL per 1 mg of rolapitant. In some embodiments, the amount of polar organic solvent is about 1.1 mL, about 1.2 mL, about 1.5 mL, about 1.7 mL, about 1.8 mL, about 1.9 mL, about 2.0 mL, about 2.2 mL, about 2.5 mL, about 2.6 mL, about 2.75 mL, about 3 mL, or about 4 mL per 1 mg of rolapitant. In some embodiments, the amount of polar organic solvent is from about 0.5 mL to about 3 mL per 1 mg of rolapitant. In some embodiments, the polar organic solvent is methanol and the concentration of rolapitant in the methanolic solution is from about 0.005 mM to about 10 mM, from about 0.05 mM to about 7 mM, from about 0.1 mM to about 5 mM, or from about 0.5 mM to about 3 mM, from about 0.5 mM to about 2 mM, from about 0.5 mM to about 2.5 mM, from about 0.6 mM to about 2 mM, or from about 0.8 mM to about 1.8 mM. In some embodiments, the polar organic solvent is methanol and the concentration of rolapitant in the methanolic solution is about 0.5 mM, about 0.6 mM, about 0.7 mM, about 0.8 mM, about 0.9 mM, about 1 mM, about 1.1 mM, about 1.2 mM, about 1.3 mM, about 1.4 mM, about 1.5 mM, about 1.6 mM, about 1.7 mM, about 1.9 mM, about 2.0 mM, about 2.1 mM, about 2.2 mM, about 2.3 mM, about 2.4 mM, or about 2.5 mM.

### Formation of the First Aqueous Solution

In some embodiments, a defined amount of human serum albumin is dissolved in an amount of water to form a first aqueous solution.

In some embodiments, the amount of aqueous solvent (e.g., water, saline, or a buffer (e.g., any one of buffers described herein)) to prepare the first aqueous solution is from about 1 mL to about 10000 L, from about 2 mL to about 1000 L, from about 3 mL to about 100 L, from about 4 mL to about 10 L, from about 5 mL to about 2 L, from about 6 mL to about 1 L.

In some embodiments, the amount of HSA prepare the first aqueous solution is from about 100 mg to about 1000 kg, from about 150 mg to about 1000 kg, from about 200 mg to about 100 kg, from about 300 mg to about 5 kg, from about 200 mg to about 500 g, or from about 200 mg to about 100 g.

In some embodiments, the amount of aqueous solvent in the first aqueous solution is from about 0.005 mL to about 10 mL per 1 mg of human serum albumin. In some embodiments, the amount of aqueous solvent in the first aqueous solution is from about 0.01 mL to about 5 mL per 1 mg of human serum albumin. In some embodiments, the amount of aqueous solvent in the first aqueous solution is from about 0.01 mL to about 1 mL per 1 mg of human serum albumin. In some embodiments, the amount of aqueous solvent in the first aqueous solution is from about 0.01 mL to about 0.5 mL per 1 mg of human serum albumin. In some embodiments, the amount of aqueous solvent in the first aqueous solution is from about 0.01 mL to about 0.1 mL per 1 mg of human serum albumin. In some embodiments, the amount of aqueous solvent in the first aqueous solution is from about 0.01 mL to about 0.05 mL per 1 mg of human serum albumin. In some embodiments, the amount of aqueous solvent in the first aqueous solution is from about 0.01 mL to about 0.03 mL per 1 mg of human serum albumin. In some embodiments, the amount of aqueous solvent in the first aqueous solution is from about 0.015 mL to about 0.04 mL per 1 mg of human serum albumin. In some embodiments, the amount of aqueous solvent in the first aqueous solution is from about 0.015 mL to about 0.025 mL per 1 mg of human serum albumin. In some embodiments, the amount of aqueous solvent in the first aqueous solution is about 0.007 mL, about 0.01 mL, about 0.011 mL. about 0.012 mL, about 0.013 mL, about 0.014 mL, about 0.015 mL, about 0.016 mL, about 0.017 mL, about 0.018 mL, about 0.019 mL, about 0.02 mL, about 0.021 mL, about 0.023 mL, about 0.025 mL, about 0.03 mL, about 0.035 mL, about 0.04 mL, about 0.045 mL, or about 0.05 mL per 1 mg of human serum albumin. In some embodiments, the amount of aqueous solvent in the first aqueous solution is about 0.02 mL per 1 mg of human serum albumin. In some embodiments, the amount of aqueous solvent (e.g., water) to prepare the first aqueous solution is from about or from about 0.005 mL to about 1 mL, from about 0.015 mL to about 0.5 mL, from about 0.015 mL to about 0.2 mL, from about 0.015 mL to about 0.1 mL, or from about 0.015 mL to about 0.05 mL per 1 mg of HSA. In some embodiments, the amount of aqueous solvent (e.g., water) to prepare the first aqueous solution is about 0.01 mL, about 0.015 mL, about 0.019 mL about 0.02 mL, about 0.021 mL, about 0.022 mL, about 0.023 mL, about 0.024 mL, about 0.025 mL, about 0.026 mL, about 0.027 mL, about 0.028 mL, about 0.029 mL or about 0.03 mL per 1 mg of HSA. In some embodiments, concentration of HSA in the first aqueous solution is from about 1 wt. % to about 20 wt. %, from about 2 wt. % to about 15 wt. %, from about 3 wt. % to about 12 wt. %, or from about 3.0 wt. % to about 10 wt. %. In some embodiments, concentration of HSA in the first aqueous solution is about 3.5 wt. %, about 3.6 wt. %, about 3.8 wt. %, about 4.0 wt. %, about 4.1 wt. %, about 4.5 wt. %, about 4.6 wt. %, about 4.7 wt. %, about 4.8 wt. %, about 4.9 wt. %, about 3.5 wt. %, about 5.0 wt. %, about 5.2 wt. %, about 5.6 wt. %, about 6.0 wt. %, about 6.5 wt. %, about 7.0 wt. %, about 8.0 wt. %, about 9.0 wt. % or about 10 wt. %.

In some embodiments, the preparation of the organic solution and the preparation of the first aqueous solution are performed concurrently.

In some embodiments, the preparation of the organic solution and the preparation of the first aqueous solution are performed sequentially. In some embodiments, the preparation of the organic solution is performed before the preparation of the first aqueous solution. In some embodiments, the preparation of the first aqueous solution is performed before the preparation of the organic solution.

In some embodiments, the range of pH in the first aqueous solution is from about 3 to about 9, from about 4 to about 8, from about 5 to about 7, from about 6 to about 7, from about 3 to about 5, from about 3 to about 7, from about 4 to about 6, or from about 6 to about 6. In some embodiments, the pH of the first aqueous solution is about 4, about 5, about 6, about 6.4, about 6.5, about 6.6, about 6.7, about 6.8, about 6.9, about 7, about 7.1, about 7.2, about 7.3, about 7.4, about 7.5, or about 8.

### Formation of the Second Aqueous Solution

In some embodiments, the organic solution of rolapitant is mixed with the first aqueous solution of human serum albumin to form a second aqueous solution. In some embodiments, the second aqueous solution is a clear aqueous solution.

In some embodiments, the volume ratio of the amount of water to the amount of the polar organic solvent is in a range from about 1:1 to about 1000:1. In some embodiments, the volume ratio of the amount of water to the amount of the polar organic solvent is in a range from about 1.5:1 to about 100:1. In some embodiments, the volume ratio of the amount of water to the amount of the polar organic solvent is in a range from about 1.5:1 to about 20:1. In some embodiments, the volume ratio of the amount of water to the amount of the polar organic solvent is in a range from about 1.5:1 to about 10:1. In some embodiments, the volume ratio of the amount of water to the amount of the polar organic solvent is in a range from about 2:1 to about 10:1, or from about 2:1 to about 3:1. In some embodiments, the volume ratio of the amount of water to the amount of the polar organic solvent is in a range from about 2:1 to about 2.5:1. In some embodiments, the volume ratio of the amount of water to the amount of the polar organic solvent is about 1.5:1, about 2:1, about 2.2:1, about 2.3:1, about 2.4:1, about 2.5:1, about 2.6:1, about 3:1, about 4:1, about 5:1, about 6:1, about 7:1, about 8:1, about 9:1, or about 10:1.

In some embodiments, the organic solution is added to the first aqueous solution to form a second aqueous solution. In some embodiments, the organic solution is added dropwise to the first aqueous solution to form a second aqueous solution. In some embodiments, the first aqueous solution is added to the organic solution to form a second aqueous solution. In some embodiments, the mixing is performed with agitation. In some embodiments, the mixing is performed with stirring. In some embodiments, the mixing is performed with shaking.

In some embodiments, the addition is done at the temperature from about 0° C. to about 35° C. In some embodiments, the addition is done at the temperature from about 0° C. to about 25° C. In some embodiments, the addition is done at the temperature from about 0° C. to about 10° C. In some embodiments, the addition is done at the temperature from about 0° C. to about 5° C. In some embodiments, the addition is done at the temperature about 0° C. In some embodiments, the addition is done at the temperature about 5° C. In some embodiments, the addition is done at the temperature about 10° C.

In some embodiments, the time of addition is in a range from about 0.1 min to about 24 hours. In some embodiments, the time of addition is in a range from about 1 min to about 2 hour. In some embodiments, the time of addition is in a range from about 1 min to about 1 hour. In some embodiments, the time of addition is in a range from about 5 min to about 30 min.

In some embodiments, the rate of addition of organic solution to the first aqueous solution is from about 0.01 mL/min to about 100 mL/min, from about 0.02 mL/min to about 50 mL/min, from about 0.05 mL/min to about 20 mL/min, from about 1 mL/min to about 10 mL/min, or from about 0.01 mL/min to about 10 mL/min, from about 0.01 mL/min to about 5 mL/min, from about 0.01 mL/min to about 2 mL/min, from about 0.01 mL/min to about 1 mL/min, from about 0.01 mL/min to about 0.5 mL/min, or from about 0.01 mL/min to about 0.1 mL/min.

In some embodiments, the rate of addition of organic solution to the first aqueous solution is about 0.01 mL/min, 0.02 mL/min, 0.03 mL/min, 0.04 mL/min, 0.05 mL/min, 0.1 mL/min, 0.2 mL/min, 0.3 mL/min, 0.5 mL/min, 0.6 mL/min, 0.8 mL/min, 1 mL/min, 1.5 mL/min, 2 mL/min, 3 mL/min, 5 mL/min or 10 mL/min.

In some embodiments, the resulting composition comprising the rolapitant and the human serum albumin can have any ratio by weight of the rolapitant to the human serum albumin as described herein. In some embodiments, the human serum albumin is a fatty acid free human serum albumin. In some embodiments, the human serum albumin is essentially fatty acid free.

In some embodiments, the range of pH in the second aqueous solution is from about 3 to about 9, from about 4 to about 8, from about 5 to about 7, from about 6 to about 7, from about 3 to about 5, from about 3 to about 7, from about 4 to about 6, or from about 6 to about 6. In some embodiments, the pH of the second aqueous solution is about 4, about 5, about 6, about 6.4, about 6.5, about 6.6, about 6.7, about 6.8, about 6.9, about 7, about 7.1, about 7.2, about 7.3, about 7.4, about 7.5, or about 8.

### Removal of Organic Solvent

In some embodiments, upon completion of mixing of the organic solution with the first aqueous solution to form the second aqueous solution, the polar organic solvent is removed from the second aqueous solution.

In some embodiments, the polar organic solvent is removed under reduced pressure. In some embodiments, the polar organic solvent is removed using rotary evaporation. In some embodiments, the polar organic solvent is removed under a vacuum.

In some embodiments, the removal of the polar organic solvent yields a clear aqueous solution. In some embodiments, water is removed from the aqueous under a vacuum. In some embodiments, water is removed from the aqueous solution using rotary evaporation. In some embodiments, water is removed from the aqueous solution by lyophilization.

In some embodiments, the solvents including both water and organic solvent are removed from the second aqueous solution simultaneously to provide a solid composition. In some embodiments, the solvents are removed under a vacuum. In some embodiments, the solvents are removed using rotary evaporation. In some embodiments, the solvents are removed by lyophilization. In some embodiments, the second aqueous solution was filtered before removal of the solvents.

Removal of Water from the Second Aqueous Solution

In some embodiments, upon removal of the organic solvent from the second aqueous solution, the water can be removed from the second aqueous solution to provide a solid composition.

In some embodiments, the second aqueous solution is filtered before removal of water. For example, the second aqueous solution can be filtered by a 0.22 micron filter before removal of water.

As used herein, the term “micron” refers to a unit of measure of one one-thousandth of a millimeter.

In some embodiments, the water is removed under a vacuum. In some embodiments, the water is removed using rotary evaporation. In some embodiments, the water is removed by lyophilization.

In some embodiments, the amount of rolapitant that is bound to the HSA (e.g., non-covalently) in the solid composition comprising the composition comprising rolapitant and HSA (as described herein) is at least 40%, at least 50%, at least 60%, at least 70%, at least 80%, at least 90%, at least 95%, at least 99%, or about 100% of the total about of rolapitant in the solid composition.

### Reconstitution of the Solid

In some embodiments the solid composition comprising the rolapitant and the human serum albumin (e.g., the solid composition prepared by removing organic solvent from the second aqueous solution and removing water from the second aqueous solution) is mixed with an aqueous solution. In some embodiments, the aqueous solution is a saline solution. In some embodiments, the aqueous solution is a 5% dextrose water solution. In some embodiments, the mixing is the addition of the aqueous solution to the solid. In some embodiments, the mixing is the addition of the solid to the aqueous solution. In some embodiments, the mixing reconstitutes the solid. In some embodiments, the mixing yields a clear aqueous solution. In some embodiments, the pH of the reconstituted solution is from about 3 to about 9, from about 4 to about 8, from about 5 to about 7, from about 6 to about 7, from about 3 to about 5, from about 3 to about 7, from about 4 to about 6, or from about 6 to about 6. In some embodiments, the pH of the reconstituted solution is about 4, about 5, about 6, about 6.4, about 6.5, about 6.6, about 6.7, about 6.8, about 6.9, about 7, about 7.1, about 7.2, about 7.3, about 7.4, about 7.5, or about 8.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

### Composition Prepared by the Process

In some embodiments, the present disclosure provides a composition comprising rolapitant and human serum albumin, wherein the rolapitant and the human serum albumin in the composition have a ratio by weight as described herein (e.g., from about 1:20 to about 1:2000), produced by a method comprising the steps of:

(i) obtaining an organic solution of rolapitant in a polar water-miscible organic solvent;

(ii) obtaining a first aqueous solution of human serum albumin; and

(iii) mixing the organic solution of rolapitant and the first aqueous solution of human serum albumin to obtain a second aqueous solution comprising the composition comprising rolapitant and human serum albumin.

In some embodiments, the rolapitant and the human serum albumin in the composition have a ratio by weight from about 1:100 to about 1:1000, from about 1:130 to about 1:800, from about 1:140 to about 1:600, about 1:140 to about 1:300, from about 1:150 to about 1:500, from about 1:160 to about 1:500, from about 1:170 to about 1:500, from about 1:180 to about 1:500, from about 1:185 to about 1:500, from about 1:190 to about 1:500, from about 1:195 to about 1:500, from about 1:200 to about 1:500, from about 1:150 to about 1:400, from about 1:160 to about 1:400, from about 1:170 to about 1:400, from about 1:180 to about 1:400, from about 1:185 to about 1:400, from about 1:190 to about 1:400, from about 1:195 to about 1:400, from about 1:200 to about 1:400, from about 1:180 to about 1:350, from about 1:185 to about 1:350, from about 1:190 to about 1:350, from about 1:195 to about 1:350, from about 1:200 to about 1:350, from about 1:185 to about 1:300, from about 1:190 to about 1:300, from about 1:195 to about 1:300, or from about 1:200 to about 1:300. In some embodiments, the rolapitant and the human serum albumin have a ratio by weight of about 1:130, about 1:140, about 1:150, about 1:160, about 1:165, about 1:170, about 1:175, about 1:180, about 185, about 1:190, about 1:195, about 1:200, about 1:205, about 1:210, about 1:215, about 1:220, about 1:225, about 1:230, about 1:235, about 1:240, about 1:245, or about 1:250, about 1:260, about 1:270, about 1:280, about 1:290, or about 1:300.

In some embodiments, the rolapitant can be a pharmaceutically acceptable salt of rolapitant. In some embodiments, rolapitant is rolapitant hydrochloride. In some embodiments, rolapitant can be any one of crystal forms, amorphous forms, solvates and hydrates as described herein. In some embodiments, rolapitant hydrochloride can be any one of crystal forms, amorphous forms, solvates and hydrates as described herein.

In some embodiments, the human serum albumin is essentially fatty acid free.

In some embodiments, the composition comprises a non-covalently bound complex comprising rolapitant and human serum albumin.

In some embodiments, the amount of polar water-miscible organic solvent is from about 0.005 mL to about 10 mL per 1 mg of rolapitant. In some embodiments, the amount of polar organic solvent is from about 0.01 mL to about 5 mL per 1 mg of rolapitant. In some embodiments, the amount of polar organic solvent is from about 0.05 mL to about 5 mL per 1 mg of rolapitant. In some embodiments, the amount of polar organic solvent is from about 0.1 mL to about 3.0 mL per 1 mg of rolapitant. In some embodiments, the amount of polar organic solvent is from about 0.2 mL to about 2.0 mL per 1 mg of rolapitant. In some embodiments, the amount of polar organic solvent is from about 0.5 mL to about 4.0 mL, from about 0.6 mL to about 3.9 mL, from about 0.7 mL to about 3.8 mL, from about 0.8 mL to about 3.7 mL, from about 0.9 mL to about 3.6 mL, from about 1.0 mL to about 3.0 mL, or from about 1.1 mL to about 2.6 mL per 1 mg of rolapitant. In some embodiments, the amount of polar organic solvent is about 1.1 mL, about 1.2 mL, about 1.5 mL, about 1.7 mL, about 1.8 mL, about 1.9 mL, about 2.0 mL, about 2.2 mL, about 2.5 mL, about 2.6 mL, about 2.75 mL, about 3 mL, or about 4 mL per 1 mg of rolapitant In some embodiments, the amount of polar organic solvent is from about 0.5 mL to about 3 mL per 1 mg of rolapitant. In some embodiments, the polar organic solvent is methanol and the concentration of rolapitant in the methanolic solution is from about 0.005 mM to about 10 mM, from about 0.05 mM to about 7 mM, from about 0.1 mM to about 5 mM, or from about 0.5 mM to about 3 mM, from about 0.5 mM to about 2 mM, from about 0.6 mM to about 2 mM, or from about 0.8 mM to about 1.8 mM. In some embodiments, the polar organic solvent is methanol and the concentration of rolapitant in the methanolic solution is about 0.6 mM, about 0.7 mM, about 0.8 mM, about 0.9 mM, about 1 mM, about 1.1 mM, about 1.2 mM, about 1.3 mM, about 1.5 mM, about 1.6 mM, about 1.7 mM, or about 1.9 mM.

In some embodiments, the amount of aqueous solvent in the first aqueous solution is from about 0.008 mL to about 0.05 mL per 1 mg of human serum albumin.

In some embodiments, the amount of aqueous solvent in the first aqueous solution is from about 0.01 mL to about 0.04 mL or from about 0.015 mL to about 0.022 mL per 1 mg of human serum albumin.

In some embodiments, the amount of aqueous solvent in the first aqueous solution is from about 0.005 mL to about 10 mL per 1 mg of human serum albumin. In some embodiments, the amount of aqueous solvent in the first aqueous solution is from about 0.01 mL to about 5 mL per 1 mg of human serum albumin. In some embodiments, the amount of aqueous solvent in the first aqueous solution is from about 0.01 mL to about 1 mL per 1 mg of human serum albumin. In some embodiments, the amount of aqueous solvent in the first aqueous solution is from about 0.01 mL to about 0.5 mL per 1 mg of human serum albumin.

In some embodiments, the amount of aqueous solvent in the first aqueous solution is from about 0.01 mL to about 0.1 mL per 1 mg of human serum albumin. In some embodiments, the amount of aqueous solvent in the first aqueous solution is from about 0.01 mL to about 0.05 mL per 1 mg of human serum albumin. In some embodiments, the amount of aqueous solvent in the first aqueous solution is from about 0.015 mL to about 0.04 mL per 1 mg of human serum albumin. In some embodiments, the amount of aqueous solvent in the first aqueous solution is from about 0.01 mL to about 0.03 mL per 1 mg of human serum albumin. In some embodiments, the amount of aqueous solvent in the first aqueous solution is about 0.007 mL, about 0.01 mL, about 0.011 mL. about 0.012 mL, about 0.013 mL, about 0.014 mL, about 0.015 mL, about 0.016 mL, about 0.017 mL, about 0.018 mL, about 0.019 mL, about 0.02 mL, about 0.021 mL, about 0.023 mL, about 0.025 mL, about 0.03 mL, about 0.035 mL, about 0.04 mL, about 0.045 mL, or about 0.05 mL per 1 mg of human serum albumin. In some embodiments, the amount of aqueous solvent (e.g., water) to prepare the first aqueous solution is from about or from about 0.005 mL to about 1 mL, from about 0.015 mL to about 0.5 mL, from about 0.015 mL to about 0.2 mL, from about 0.015 mL to about 0.1 mL, or from about 0.015 mL to about 0.05 mL per 1 mg of HSA. In some embodiments, the amount of aqueous solvent (e.g., water) to prepare the first aqueous solution is about 0.01 mL, about 0.015 mL, about 0.019 mL about 0.02 mL, about 0.021 mL, about 0.022 mL, about 0.023 mL, about 0.024 mL, about 0.025 mL, about 0.026 mL, about 0.027 mL, about 0.028 mL, about 0.029 mL or about 0.03 mL per 1 mg of HSA. In some embodiments, the amount of aqueous solvent in the first aqueous solution about 0.02 mL per 1 mg of human serum albumin. In some embodiments, concentration of HSA in the first aqueous solution is from about 1 wt. % to about 20 wt. %, from about 2 wt. % to about 15 wt. %, from about 3 wt. % to about 12 wt. %, or from about 3.0 wt. % to about 10 wt. %. In some embodiments, concentration of HSA in the first aqueous solution is about 3.5 wt. %, about 3.6 wt. %, about 3.8 wt. %, about 4.0 wt. %, about 4.1 wt. %, about 4.5 wt. %, about 4.6 wt. %, about 4.7 wt. %, about 4.8 wt. %, about 4.9 wt. %, about 3.5 wt. %, about 5.0 wt. %, about 5.2 wt. %, about 5.6 wt. %, about 6.0 wt. %, about 6.5 wt. %, about 7.0 wt. %, about 8.0 wt. %, about 9.0 wt. % or about 10 wt. %.

In some embodiments, the polar water-miscible organic solvent is an alcohol selected from the group consisting of methanol, ethanol, isopropanol, n-butanol, and mixtures thereof.

In some embodiments, the polar water-miscible organic solvent is selected from methanol, ethanol, and mixtures thereof.

In some embodiments, the polar water-miscible organic solvent is methanol.

In some embodiments, the aqueous solvent is water.

In some embodiments, the polar water-miscible organic solvent is methanol and the aqueous solvent in the first aqueous solution is water.

In some embodiments, the mixing comprises adding the organic solution to the first aqueous solution. In some embodiments, wherein the mixing comprises adding the first aqueous solution to the organic solution. In some embodiments, the adding is carried out dropwise. In some embodiments, the adding is carried out for a period of time from several minutes to several hours. In some embodiments, the adding is carried out for a period of time from 2 min to 24 hours. In some embodiments, the adding is carried out for a period of time from 2 min minutes to 12 hours, from 2 min to 6 hours, from 3 min to 3 hours, from 2 min to 1 hour, from 2 min to 30 min, or from 2 min to 25 min.

In some embodiments, the rate of addition of organic solution to the first aqueous solution is from about 0.01 mL/min to about 100 mL/min, from about 0.02 mL/min to about 50 mL/min, from about 0.05 mL/min to about 20 mL/min, from about 1 mL/min to about 10 mL/min, or from about 0.01 mL/min to about 10 mL/min, from about 0.01 mL/min to about 5 mL/min, from about 0.01 mL/min to about 2 mL/min, from about 0.01 mL/min to about 1 mL/min, from about 0.01 mL/min to about 0.5 mL/min, or from about 0.01 mL/min to about 0.1 mL/min.

In some embodiments, the rate of addition of organic solution to the first aqueous solution is about 0.01 mL/min, 0.02 mL/min, 0.03 mL/min, 0.04 mL/min, 0.05 mL/min, 0.1 mL/min, 0.2 mL/min, 0.3 mL/min, 0.5 mL/min, 0.6 mL/min, 0.8 mL/min, 1 mL/min, 1.5 mL/min, 2 mL/min, 3 mL/min, 5 mL/min or 10 mL/min.

In some embodiments, the mixing is carried out at a temperature from about 0° C. to about 25° C. In some embodiments, mixing is carried out at ambient temperature (e.g., about 25° C.). In some embodiments, the mixing is carried out at a temperature from about 0° C. to about 5° C. In some embodiments, the mixing is carried out at about 0° C.

In some embodiments, the volume ratio of the amount of aqueous solvent to the amount of the organic solvent in the second aqueous solution is in a range from about 1:1 to about 1000:1. In some embodiments, the volume ratio of the amount of aqueous solvent to the amount of the organic solvent in the second aqueous solution is in a range from about 1.5:1 to about 100:1. In some embodiments, the volume ratio of the amount of aqueous solvent to the amount of the organic solvent in the second aqueous solution is in a range from about 1.5:1 to about 20:1. In some embodiments, the volume ratio of the amount of aqueous solvent to the amount of the organic solvent in the second aqueous solution is in a range from about 1.5:1 to about 10:1. In some embodiments, the volume ratio of the amount of aqueous solvent to the amount of the organic solvent in the second aqueous solution is in a range from about 2:1 to about 10:1 or from about 2:1 to about 3:1. In some embodiments, the volume ratio of the amount of aqueous solvent to the amount of the organic solvent in the second aqueous solution is about 1.5:1, about 2:1, about 2.2:1, about 2.3:1, about 2.4:1, about 2.5:1, about 2.6:1, about 3:1, about 4:1, about 5:1, about 6:1, about 7:1, about 8:1, about 9:1, or about 10:1. In some embodiments, the aqueous solvent is water. In some embodiments, the aqueous solvent is water and the organic solvent is an alcohol. In some embodiments, the aqueous solvent is water and the organic solvent is methanol.

In some embodiments, the composition further comprises removing the polar water-miscible organic solvent from the second aqueous solution to obtain a third aqueous solution comprising the composition comprising rolapitant and human serum albumin. In some embodiments, the composition comprises removing aqueous solvent from the third aqueous solution to obtain the composition comprising rolapitant and human serum albumin.

In some embodiments, the composition further comprises removing the organic solvent (e.g. methanol) and the aqueous solvent (e.g., water) from the second aqueous solution to obtain the composition comprising rolapitant and human serum albumin.

In some embodiments, the removing as carried out in vacuum (e.g., using the rotovap). In some embodiments, the removing is carried out by lyophilization.

In some embodiments, the composition forms a clear aqueous solution when the composition is dissolved in an aqueous solvent, and wherein the solubility of the composition in the aqueous solution is at least 20 mg/ml.

In some embodiments, the composition is a solid formulation

In some embodiments, the composition is an aqueous formulation. In some embodiments, the aqueous formulation is substantially free of solvent other than water. In some embodiments, the aqueous formulation is free of a surfactant. In some embodiments, the surfactant is selected from the group consisting of CREMOPHOR® surfactants and Polysorbate 80.

In some embodiments, the aqueous formulation is a clear aqueous solution. In some embodiments, the aqueous formulation is a clear aqueous solution for at least 1 hour, at least 2 hours, at least 3 hours, at least 4 hours, at least 5 hours, at least 6 hours, at least 8 hours, or at least 24 hours.

In some embodiments, the present disclosure provides a pharmaceutical composition comprising the composition as prepared by a process as described herein, and a pharmaceutically acceptable carrier.

In some embodiments, the present disclosure provides a method for the prevention of chemotherapy-induced nausea and vomiting or postoperative nausea and vomiting (PONV), the method comprising the step of administering to a subject in need thereof of a therapeutically effective amount of a pharmaceutical composition comprising the composition comprising the rolapitant and the human serum albumin as described herein, and a pharmaceutically acceptable carrier.

### EXAMPLES

### Materials and Methods

Hplc Analysis:

The HPLC system used herein is a SHIMADZU LC-10AT vp series system, which consists of a SHIMADZU LC-10AT vp pump, a manual injector, a SHIMADZU CTO-10AS vp column oven, a SHIMADZU SPD-10A vp wavelength detector, and a SHIMADZU LC solution workstation. Agilent Zorbax XDB-C18 column (4.6 mm×50 mm, 5 μm) is used as an analytical HPLC column. Mobile phases A and B consist of water with 0.1% trifluoroacetic acid (TFA) and methanol with 0.1% TFA, respectively. The mobile phases were delivered at a programmed linear gradient. Separation was pumped at a flow rate of 1 ml/minute, and initiated and maintained at a mobile phase ratio of 85:15 (A:B) for iminute.

The ratio was changed to 10:90 (A:B) over a period of 2 minutes using a linear curve and then maintained at 10:90 (A:B) over a period of 4.5 minutes. The mobile phase was subsequently changed back to 85:15 (A:B) over a period of 1 minute and this ratio was maintained for 1.5 minutes before the next sample was injected. The effluent is detected at a wavelength of 254 nm using a UV detector. The sample injection amount is 20 μl.

### Example 1: Composition Comprising Rolapitant and Human Serum Albumin (HSA)

The Ratio by Weight of Rolapitant to HSA Prepared was about 1:140.

Rolapitant (2 mg) was dissolved in methanol (3 ml) in a vial to give a clear solution.

HSA (280 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a cloudy solution.

### Example 2: Composition Comprising Rolapitant and Human Serum Albumin (HSA)

The Ratio by Weight of Rolapitant to HSA Prepared was about 1:150.

Rolapitant (2 mg) was dissolved in methanol (3 ml) in a vial to give a clear solution.

HSA (300 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a cloudy solution.

### Example 3: Composition Comprising Rolapitant and Human Serum Albumin (HSA)

The Ratio by Weight of Rolapitant to HSA Prepared was about 1:200.

Rolapitant (2 mg) was dissolved in methanol (3.4 ml) in a vial to give a clear solution.

HSA (400 mg) (native fatty acid free human serum albumin purchased from SeraCare Life Sciences, product code: HS-455-80, which contains fatty acids <0.2 mg/gm) as a powder was dissolved in 8 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a clear solution.

### Example 4: Composition Comprising Rolapitant and Human Serum Albumin (HSA)

The Ratio by Weight of Rolapitant to HSA Prepared was about 1:165.

Rolapitant (2 mg) was dissolved in methanol (3 ml) in a vial to give a clear solution. HSA (330 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a cloudy solution with some precipitation.

### Example 5: Composition Comprising Rolapitant and Human Serum Albumin (HSA)

The Ratio by Weight of Rolapitant to HSA Prepared was about 1:170.

Rolapitant (2 mg) was dissolved in methanol (3 ml) in a vial to give a clear solution. HSA (340 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a cloudy solution with some precipitation.

### Example 6: Composition Comprising Rolapitant and Human Serum Albumin (HSA)

The Ratio by Weight of Rolapitant to HSA Prepared was about 1:180.

Rolapitant (2 mg) was dissolved in methanol (3 ml) in a vial to give a clear solution. HSA (360 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a cloudy solution with some precipitation.

### Example 7: Composition Comprising Rolapitant and Human Serum Albumin (HSA)

The Ratio by Weight of Rolapitant to HSA Prepared was about 1:190.

Rolapitant (2 mg) was dissolved in methanol (3 ml) in a vial to give a clear solution. HSA (380 mg) (native fatty acid free human serum albumin purchased from SeraCare Life Sciences, product code: HS-455-80, which contains fatty acids <0.2 mg/gm) as a powder was dissolved in 7 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a slightly cloudy solution.

### Example 8: Composition Comprising Rolapitant and Human Serum Albumin (HSA)

The Ratio by Weight of Rolapitant to HSA Prepared was about 1:300.

Rolapitant (2 mg) was dissolved in methanol (5.1 ml) in a vial to give a clear solution. HSA (600 mg) (native fatty acid free human serum albumin purchased from SeraCare Life Sciences, product code: HS-455-80, which contains fatty acids <0.2 mg/gm) as a powder was dissolved in 12 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature.

### Example 9: Composition Comprising Rolapitant and Human Serum Albumin (HSA)

The Ratio by Weight of Rolapitant to HSA Prepared was about 1:200.

Rolapitant (1 mg) was dissolved in methanol (1.7 ml) in a vial to give a clear solution. HSA (200 mg) (native fatty acid free human serum albumin purchased from SeraCare Life Sciences, product code: HS-455-80, which contains fatty acids <0.2 mg/gm) as a powder was dissolved in 4 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature.

### Example 10: Composition Comprising Rolapitant and Human Serum Albumin (HSA)

The Ratio by Weight of Rolapitant to HSA Prepared was about 1:220.

Rolapitant (1 mg) was dissolved in methanol (1.7 ml) in a vial to give a clear solution. HSA (220 mg) (native fatty acid free human serum albumin purchased from SeraCare Life Sciences, product code: HS-455-80, which contains fatty acids <0.2 mg/gm) as a powder was dissolved in 4 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature.

### Example 11: Composition Comprising Rolapitant and Human Serum Albumin (HSA)

The Ratio by Weight of Rolapitant to HSA Prepared was about 1:200.

Rolapitant (1 mg) was dissolved in methanol (1.7 ml) in a vial to give a clear solution. A solution of HSA (200 mg, 1 ml) (20% human serum albumin solution for infusion (product name: AlbuRx) from CSL Behring) was added into 3 ml of water to give a HSA solution (4 ml) in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a cloudy solution with some precipitation.

### Example 12: Composition Comprising Rolapitant and Human Serum Albumin (HSA)

The Ratio by Weight of Rolapitant to HSA Prepared was about 1:250.

Rolapitant (1 mg) was dissolved in methanol (1.7 ml) in a vial to give a clear solution. A solution of HSA (250 mg, 1.25 ml) (20% human serum albumin solution for infusion (product name: AlbuRx) from CSL Behring) was added into 3 ml of water to give a HSA solution (4.25 ml) in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature. This aqueous solution stays clear with no precipitation after 6 hours at room temperature.

### Example 13: Composition Comprising Rolapitant and Human Serum Albumin (HSA)

The Ratio by Weight of Rolapitant to HSA Prepared was about 1:280.

Rolapitant (1 mg) was dissolved in methanol (1.7 ml) in a vial to give a clear solution.

A solution of HSA (280 mg, 1.4 ml) (20% human serum albumin solution for infusion (product name: AlbuRx) from CSL Behring) was added into 3 ml of water to give a HSA solution (4.4 ml) in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The resulting clear aqueous solution was lyophilized overnight to give a white solid.

A sample of 100 mg of the lyophilized solid was reconstituted by adding 2 mL water to give a clear solution. This aqueous solution stays clear with no precipitation after 1 hour at room temperature. This aqueous solution stays clear with no precipitation after 2 hours at room temperature. This aqueous solution stays clear with no precipitation after 3 hours at room temperature. This aqueous solution stays clear with no precipitation after 4 hours at room temperature. This aqueous solution stays clear with no precipitation after 5 hours at room temperature. This aqueous solution stays clear with no precipitation after 6 hours at room temperature.

### Example 14: Composition Comprising Rolapitant and Human Serum Albumin (HSA)

The Ratio by Weight of Rolapitant to HSA Prepared was about 1:200.

Rolapitant (15 mg) was dissolved in methanol (17.2 ml) in a flask to give a clear solution. HSA (3000 mg) (native fatty acid free human serum albumin purchased from SeraCare Life Sciences, product code: HS-455-80, which contains fatty acids <0.2 mg/gm) as a powder was dissolved in 40 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution.

### Example 15: Composition Comprising Rolapitant and Human Serum Albumin (HSA)

The Ratio by Weight of Rolapitant to HSA Prepared was about 1:220.

Rolapitant (10 mg) was dissolved in methanol (18.8 ml) in a flask to give a clear solution. HSA (2200 mg) (native fatty acid free human serum albumin purchased from SeraCare Life Sciences, product code: HS-455-80, which contains fatty acids <0.2 mg/gm) as a powder was dissolved in 44 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution.

### Example 16: Composition Comprising Rolapitant and Human Serum Albumin (HSA)

The Ratio by Weight of Rolapitant to HSA Prepared was about 1:210.

Rolapitant (10 mg) was dissolved in methanol (12 ml) in a flask to give a clear solution. HSA (2100 mg) (native fatty acid free human serum albumin purchased from SeraCare Life Sciences, product code: HS-455-80, which contains fatty acids <0.2 mg/gm) as a powder was dissolved in 28 ml of water in a round bottom flask. The methanol solution of rolapitant was added slowly dropwise into the flask of the HSA solution with rapid stirring at 0° C. Upon completion of the addition, a clear solution was obtained. Then, the methanol in the solution was removed under vacuum to give a clear solution. The clear aqueous solution was filtered by a 0.22 micron aqueous phase filter. The resulting clear aqueous solution was lyophilized overnight to give a white solid.

A sample of 100 mg of the lyophilized solid was reconstituted by adding 2 mL water to give a clear solution.

### Example 17: Measuring pH Value of the Clear Aqueous Solution Comprising Composition Comprising Rolapitant and Human Serum Albumin (HSA)

500 mg of the lyophilized solid comprising the composition comprising rolapitant and HSA (the ratio by weight about 1:200) from Example 14 was dissolved in 10 ml of water to give a clear aqueous solution, which was kept at about 25° C. and measured for pH value. The pH value of the clear aqueous solution is 6.79 (3 measurement points: 6.80, 6.79, and 6.79).

### Example 18: Measuring pH Value of the Clear Aqueous Solution Comprising Composition Comprising Rolapitant and Human Serum Albumin (HSA)

250 mg of the lyophilized solid comprising the composition comprising rolapitant and HSA (the ratio by weight about 1:220) from Example 15 was dissolved in 10 ml of 0.9% saline solution, which had pH value about 5.41, to give a clear aqueous solution, which was kept at about 25° C. and measured for pH value. The pH value of the clear aqueous solution is 6.81 (3 measurement points: 6.82, 6.81, and 6.81).

### Example 19: Measuring pH Value of the Clear Aqueous Solution Comprising Composition Comprising Rolapitant and Human Serum Albumin (HSA)

250 mg of the lyophilized solid comprising the composition comprising rolapitant and HSA (the ratio by weight about 1:210) from Example 16 was dissolved in 10 ml of 0.9% saline solution, which had pH value about 5.41, to give a clear aqueous solution, which was kept at about 25° C. and measured for pH value. The pH value of the clear aqueous solution is 6.73 (3 measurement points: 6.73, 6.73, and 6.73).

500 mg of the lyophilized solid comprising the composition comprising rolapitant and HSA (the ratio by weight about 1:210) from Example 16 was dissolved in 10 ml of 0.9% saline solution, which had pH value about 5.41, to give a clear aqueous solution, which was kept at about 25° C. and measured for pH value. The pH value of the clear aqueous solution is 6.77 (3 measurement points: 6.76, 6.77, and 6.77).

250 mg of the lyophilized solid comprising the composition comprising rolapitant and HSA (the ratio by weight about 1:210) from Example 16 was dissolved in 10 ml of 5% Dextrose water solution, which had pH value about 4.40, to give a clear aqueous solution, which was kept at about 25° C. and measured for pH value. The pH value of the clear aqueous solution is 6.74 (3 measurement points: 6.74, 6.75, and 6.74).

### Example 20: Measuring the Correlation Between HPLC Peak Area and the Rolapitant Concentration

Methanol solutions of rolapitant in 6 different concentrations, 0.075 mg/ml, 0.1 mg/ml, 0.15 mg/ml, 0.2 mg/ml, 0.3 mg/ml and 0.4 mg/ml, were prepared. The 6 rolapitant methanol solutions were analyzed in HPLC. The peak area and concentration of rolapitant were correlated using linear regression. The linear regression data is shown as below.

Y(peak area)=8267+1.07538E6*X(concentration), R=0.9999, P<0.0001.

### Example 21: Measure the Rolapitant Concentrations in the Clear Aqueous Solutions Before and after the Filtration at 0 Hour, and after the Filtration at 1, 2, 3, and 24 Hours

1700 mg of the lyophilized solid of the composition comprising rolapitant and HSA from Example 14 (The ratio by weight of rolapitant to HSA is about 1:200) was dissolved in 17 ml of water to give a clear aqueous solution, which was kept at about 25° C. Immediately after the lyophilized solid was dissolved in water, 4 ml of the clear aqueous solution was taken out from the 17 ml solution. Then 1 ml of the solution was taken out from the 4 ml clear aqueous solution to give the solution RO-0-0h, and the remaining 3 ml of the solution was filtered by the same 0.22 micron aqueous phase filter at 1 ml at a time to give the solutions RO-1-0h, RO-2-0h, and RO-3-0h. To 300 μl of the solutions RO-0-0h and RO-3-0h were added 700 μl of acetonitrile separately. The mixtures were vortexed for seconds and then centrifuged at 4,000 g for 5 minutes. The supernatants were removed and collected followed by injection on HPLC. The same procedure was repeated one more time for each of solutions RO-0-0h and RO-3-0h. Based on the HPLC data and the measurement data of Example 20, the rolapitant concentrations of the solutions of RO-0-0h and RO-3-0h have been calculated and shown in the Table 1. At 0 hour, the rolapitant concentration of the clear aqueous solution after the filtration was about 95.31% of the rolapitant concentration of the clear aqueous solution before the filtration.

TABLE 1

| | Rolapitant | Average Rolapitant |
|---|---|---|
| Solution Number | Concentration (mg/ml) | Concentration (mg/ml) |
| RO-0-0 h-1 | 0.4998 | 0.4994 |
| RO-0-0 h-2 | 0.4989 | |
| RO-3-0 h-1 | 0.4764 | 0.4760 |
| RO-3-0 h-2 | 0.4756 | |

At 1 hour, 3 ml of the clear aqueous solution was taken out from the remaining 13 ml of the aqueous solution. Then 1 ml of the solution was taken out from the 3 ml clear aqueous solution and filtered by a 0.22 micron aqueous phase filter to give the solution RO-1-1h, and the remaining 2 ml of the solution was filtered by the same 0.22 micron aqueous phase filter at 1 ml at a time to give the solutions RO-2-1h and RO-3-1h. To 300 μl of the solution RO-3-1h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-1h. Based on the HPLC data and the measurement data of Example 20, the rolapitant concentrations of the solution RO-3-1h have been calculated and shown in the Table 2. At 1 hour, the rolapitant concentration of the clear aqueous solution after the filtration was about 95.77% of the rolapitant concentration of the clear aqueous solution at 0 hour before the filtration.

TABLE 2

| | Rolapitant | Average Rolapitant |
|---|---|---|
| Solution Number | Concentration (mg/ml) | Concentration (mg/ml) |
| RO-3-1 h-1 | 0.4780 | 0.4783 |
| RO-3-1 h-2 | 0.4785 | |

At 2 hours, 3 ml of the clear aqueous solution was taken out from the remaining 10 ml of the aqueous solution. Then 1 ml of the solution was taken out from the 3 ml clear aqueous solution and filtered by a 0.22 micron aqueous phase filter to give the solution RO-1-2h, and the remaining 2 ml of the solution was filtered by the same 0.22 micron aqueous phase filter at 1 ml at a time to give the solutions RO-2-2h and RO-3-2h. To 300 μl of the solution RO-3-2h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-2h. Based on the HPLC data and the measurement data of sample 20, the rolapitant concentrations of the solution RO-3-2h have been calculated and shown in the Table 3. At 2 hours, the rolapitant concentration of the clear aqueous solution after the filtration was about 95.33% of the rolapitant concentration of the clear aqueous solution at 0 hour before the filtration.

TABLE 3

| | Rolapitant | Average Rolapitant |
|---|---|---|
| Solution Number | Concentration (mg/ml) | Concentration (mg/ml) |
| RO-3-2 h-1 | 0.4746 | 0.4761 |
| RO-3-2 h-2 | 0.4776 | |

At 3 hours, 3 ml of the clear aqueous solution was taken out from the remaining 7 ml of the aqueous solution. Then 1 ml of the solution was taken out from the 3 ml clear aqueous solution and filtered by a 0.22 micron aqueous phase filter to give the solution RO-1-3h, and the remaining 2 ml of the solution was filtered by the same 0.22 micron aqueous phase filter at 1 ml at a time to give the solutions RO-2-3h and RO-3-3h. To 300 μl of the solution RO-3-3h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-3h. Based on the HPLC data and the measurement data of sample 20, the rolapitant concentrations of the solution RO-3-3h have been calculated and shown in the Table 4. At 3 hours, the rolapitant concentration of the clear aqueous solution after the filtration was about 95.59% of the rolapitant concentration of the clear aqueous solution at 0 hour before the filtration.

TABLE 4

| | Rolapitant | Average Rolapitant |
|---|---|---|
| Solution Number | Concentration (mg/ml) | Concentration (mg/ml) |
| RO-3-3 h-1 | 0.4770 | 0.4774 |
| RO-3-3 h-2 | 0.4778 | |

At 24 hours, 3 ml of the clear aqueous solution was taken out from the remaining 4 ml of the aqueous solution. Then 1 ml of the solution was taken out from the 3 ml clear aqueous solution and filtered by a 0.22 micron aqueous phase filter to give the solution RO-1-24h, and the remaining 2 ml of the solution was filtered by the same 0.22 micron aqueous phase filter at 1 ml at a time to give the solutions RO-2-24h and RO-3-24h. To 300 μl of the solution RO-3-24h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-24h. Based on the HPLC data and the measurement data of sample 20, the rolapitant concentrations of the solution RO-3-24h have been calculated and shown in the Table 5. At 24 hours, the rolapitant concentration of the clear aqueous solution after the filtration was about 96.04% of the rolapitant concentration of the clear aqueous solution at 0 hour before the filtration.

TABLE 5

| | Rolapitant | Average Rolapitant |
|---|---|---|
| Solution Number | Concentration (mg/ml) | Concentration (mg/ml) |
| RO-3-24 h-1 | 0.4793 | 0.4796 |
| RO-3-24 h-2 | 0.4798 | |

### Example 22: Measure the Rolapitant Concentrations in the Clear Aqueous Solutions Before and after the Filtration at 0 Hour, and after the Filtration at 1, 2, and 3 Hours

1500 mg of the lyophilized solid of the composition comprising rolapitant and HSA from Example 15 (The ratio by weight of rolapitant to HSA is about 1:220) was dissolved in 15 ml of water to give a clear aqueous solution, which was kept at about 25° C. Immediately after the lyophilized solid was dissolved in water, 4 ml of the clear aqueous solution was taken out from the 15 ml solution. Then 1 ml of the solution was taken out from the 4 ml clear aqueous solution to give the solution RO-0-0h, and the remaining 3 ml of the solution was filtered by the same 0.22 micron aqueous phase filter at 1 ml at a time to give the solutions RO-1-0h, RO-2-0h, and RO-3-0h. To 300 μl of the solutions RO-0-0h and RO-3-0h were added 700 μl of acetonitrile separately. The mixtures were vortexed for seconds and then centrifuged at 4,000 g for 5 minutes. The supernatants were removed and collected followed by injection on HPLC. The same procedure was repeated one more time for each of solutions RO-0-0h and RO-3-0h. Based on the HPLC data and the measurement data of Example 20, the rolapitant concentrations of the solutions of RO-0-0h and RO-3-0h have been calculated and shown in the Table 6. At 0 hour, the rolapitant concentration of the clear aqueous solution after the filtration was about 96.65% of the rolapitant concentration of the clear aqueous solution before the filtration.

TABLE 6

| | Rolapitant | Average Rolapitant |
|---|---|---|
| Solution Number | Concentration (mg/ml) | Concentration (mg/ml) |
| RO-0-0 h-1 | 0.4062 | 0.4085 |
| RO-0-0 h-2 | 0.4108 | |
| RO-3-0 h-1 | 0.3964 | 0.3948 |
| RO-3-0 h-2 | 0.3931 | |

At 1 hour, 3 ml of the clear aqueous solution was taken out from the remaining 11 ml of the aqueous solution. Then 1 ml of the solution was taken out from the 3 ml clear aqueous solution and filtered by a 0.22 micron aqueous phase filter to give the solution RO-1-1h, and the remaining 2 ml of the solution was filtered by the same 0.22 micron aqueous phase filter at 1 ml at a time to give the solutions RO-2-1h and RO-3-1h. To 300 μl of the solution RO-3-1h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-1h. Based on the HPLC data and the measurement data of Example 20, the rolapitant concentrations of the solution RO-3-1h have been calculated and shown in the Table 7. At 1 hour, the rolapitant concentration of the clear aqueous solution after the filtration was about 96.94% of the rolapitant concentration of the clear aqueous solution at 0 hour before the filtration.

TABLE 7

| | Rolapitant | Average Rolapitant |
|---|---|---|
| Solution Number | Concentration (mg/ml) | Concentration (mg/ml) |
| RO-3-1 h-1 | 0.3970 | 0.3960 |
| RO-3-1 h-2 | 0.3950 | |

At 2 hours, 3 ml of the clear aqueous solution was taken out from the remaining 8 ml of the aqueous solution. Then 1 ml of the solution was taken out from the 3 ml clear aqueous solution and filtered by a 0.22 micron aqueous phase filter to give the solution RO-1-2h, and the remaining 2 ml of the solution was filtered by the same 0.22 micron aqueous phase filter at 1 ml at a time to give the solutions RO-2-2h and RO-3-2h. To 300 μl of the solution RO-3-2h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-2h. Based on the HPLC data and the measurement data of sample 20, the rolapitant concentrations of the solution RO-3-2h have been calculated and shown in the Table 8. At 2 hours, the rolapitant concentration of the clear aqueous solution after the filtration was about 96.77% of the rolapitant concentration of the clear aqueous solution at 0 hour before the filtration.

TABLE 8

| | Rolapitant | Average Rolapitant |
|---|---|---|
| Solution Number | Concentration (mg/ml) | Concentration (mg/ml) |
| RO-3-2 h-1 | 0.3951 | 0.3953 |
| RO-3-2 h-2 | 0.3955 | |

At 3 hours, 3 ml of the clear aqueous solution was taken out from the remaining 5 ml of the aqueous solution. Then 1 ml of the solution was taken out from the 3 ml clear aqueous solution and filtered by a 0.22 micron aqueous phase filter to give the solution RO-1-3h, and the remaining 2 ml of the solution was filtered by the same 0.22 micron aqueous phase filter at 1 ml at a time to give the solutions RO-2-3h and RO-3-3h. To 300 μl of the solution RO-3-3h was added 700 μl of acetonitrile. The mixture was vortexed for seconds and then centrifuged at 4,000 g for 5 minutes. The supernatant was removed and collected followed by injection on HPLC. The same procedure was repeated one more time for the solution RO-3-3h. Based on the HPLC data and the measurement data of sample 20, the rolapitant concentrations of the solution RO-3-3h have been calculated and shown in the Table 9. At 3 hours, the rolapitant concentration of the clear aqueous solution after the filtration was about 96.62% of the rolapitant concentration of the clear aqueous solution at 0 hour before the filtration.

TABLE 9

| | Rolapitant | Average Rolapitant |
|---|---|---|
| Solution Number | Concentration (mg/ml) | Concentration (mg/ml) |
| RO-3-3 h-1 | 0.3951 | 0.3947 |
| RO-3-3 h-2 | 0.3943 | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising rolapitant and human serum albumin, wherein the rolapitant and the human serum albumin in the composition have a ratio by weight from about 1:20 to about 1:2000.

2. The composition of claim 1, wherein the rolapitant and the human serum albumin in the composition have a ratio by weight from about 1:100 to about 1:1000.

3. The composition of claim 1, wherein the rolapitant and the human serum albumin in the composition have a ratio by weight from about 1:150 to about 1:500.

4. The composition of claim 1, wherein the rolapitant and the human serum albumin in the composition have a ratio by weight from about 1:140 to about 1:300.

5. The composition of claim 1, wherein the human serum albumin is essentially fatty acid free.

6. The composition of claim 1, wherein the composition is a solid formulation.

7. The composition of claim 1, wherein the composition is an aqueous formulation.

8. The composition of claim 7, wherein the aqueous formulation is substantially free of solvent other than water.

9. The composition of claim 7, wherein the aqueous formulation is a clear aqueous solution.

10. The composition of claim 7, wherein the aqueous formulation is a clear aqueous solution for at least 2 hours.

11. The composition of claim 7, wherein the aqueous formulation is a clear aqueous solution for at least 4 hours.

12. The composition of claim 7, wherein the aqueous formulation has pH value from about 5 to about 8.

13. A composition comprising a non-covalently bound complex comprising rolapitant and human serum albumin, wherein the rolapitant and the human serum albumin in the composition have a ratio by weight from about 1:20 to about 1:2000.

14. The composition of claim 13, wherein the rolapitant and the human serum albumin in the composition have a ratio by weight from about 1:100 to about 1:1000.

15. A pharmaceutical composition comprising the composition of claim 1, and a pharmaceutically acceptable carrier.

16. The pharmaceutical composition of claim 15, wherein the composition is free of a vehicle selected from the group consisting of water soluble organic solvents, non-ionic surfactants, water insoluble lipids, organic lipids/semisolids and phospholipids.

17. A method for the prevention of chemotherapy-induced nausea and vomiting or postoperative nausea and vomiting (PONV), the method comprising the step of administering to a subject in need thereof of a therapeutically effective amount of a pharmaceutical composition of claim 15.

18. The method of claim 17, wherein the method is for the prevention of chemotherapy-induced nausea and vomiting.

19. The method of claim 17, wherein the method is for the prevention of delayed nausea and vomiting associated with initial and repeat courses of emetogenic cancer chemotherapy, including, but not limited to, highly emetogenic cancer chemotherapy (HEC).

20. A composition comprising rolapitant and human serum albumin, wherein the rolapitant and the human serum albumin in the composition have a ratio by weight from about 1:20 to about 1:2000, produced by a method comprising the steps of:
(i) obtaining an organic solution of rolapitant in a polar water-miscible organic solvent;
(ii) obtaining a first aqueous solution of human serum albumin; and
(iii) mixing the organic solution of rolapitant and the first aqueous solution of human serum albumin to obtain a second aqueous solution comprising the composition comprising rolapitant and human serum albumin.
